(19) United States
(12) Patent Application Publication
(10) Pub. No.: US 2025/0253048 A1
(43) Pub. Date: Aug. 7, 2025

(54) MACHINE LEARNING BASED DISEASE OR CONDITION PREDICTION

(71) Applicant: Culmination Bio, Inc., St. George, UT (US)

(72) Inventors: David Allen Jones, Dammeron Valley, UT (US); Lincoln Dyreng Nadauld, Santa Clara, UT (US); Richard Glenn Cua Dela Cruz, St. George, UT (US); Imelda Tumanan Sandoval, St. George, UT (US); Si Yang Ke, Lehi, UT (US); Oren Eliezer Livne, Churchville, PA (US)

(21) Appl. No.: 19/043,257

(22) Filed: Jan. 31, 2025

Related U.S. Application Data
(60) Provisional application No. 63/549,372, filed on Feb. 2, 2024.
(60) Provisional application No. 63/567,807, filed on Mar. 20, 2024.

(51) Int. Cl. G16H 50/20; G16H 10/60; G16H 50/30

(52) U.S. Cl. CPC G16H 50/20; G16H 10/60; G16H 50/30

(57) ABSTRACT

The system uses machine learning to predict diseases or conditions and assess disease risk progression from routine healthcare test results, either at a single time point or across multiple time points. The system predicts the presence or absence of a disease or condition, including screening. Routine healthcare test results include a number of typical clinical measures (such as forty, fifty, sixty measures) each with a separate value. The system makes predictions regarding diseases or conditions. The system is configured to estimate disease probability, stratify patients by risk level, and generate risk trajectories over time. The framework is designed to be extensible to future disease categories, novel biomarkers, and evolving machine learning models. The methodology applies to any disease or condition where blood-based, genetic, imaging, environmental, or real-time physiological data provide diagnostic insights.

### INCORPORATION BY REFERENCE TO ANY PRIORITY APPLICATIONS

This application claims benefit of U.S. Provisional Patent Application Ser. No. 63/549,372 entitled “MACHINE LEARNING BASED DISEASE OR CONDITION PREDICTION” filed Feb. 2, 2024, and U.S. Provisional Patent Application Ser. No. 63/567,807 entitled “MACHINE LEARNING BASED DISEASE OR CONDITION PREDICTION” filed Mar. 20, 2024, which are hereby incorporated by reference in their entiretics.

Any and all applications for which a foreign or domestic priority claim is identified in the Application Data Sheet as filed with the present application are hereby incorporated by reference under 37 CFR 1.57.

### BACKGROUND

Many existing healthcare tests look for a specific indicator (such as the A1C test that measures red blood cells that have glucose-coated hemoglobin) and a clinician can use the test results to make a specific diagnosis. For example, the A1C test (also known as the hemoglobin A1C or HbA1c test) is a blood test that measures a patient's average blood sugar levels over the past three months. The A1C test is commonly used to diagnose prediabetes and diabetes. The A1C test measures the percentage of the patient's red blood cells that have glucose-coated hemoglobin. An A1C test can show an average glucose level for the past three months because glucose sticks to hemoglobin for as long as the red blood cells are alive and red blood cells live approximately three months. Higher A1C levels are linked to diabetes complications.

A blood test or panel is a common test that healthcare providers use to monitor overall health. For example, a patient may get a blood test as part of a routine physical examination. Some tests focus on blood cells and platelets. Some tests evaluate substances in blood such as electrolytes, proteins, and hormones. Some tests measure certain minerals in blood.

### SUMMARY

The systems, methods, and devices described herein each have several aspects, no single one of which is solely responsible for its desirable attributes. Without limiting the scope of this disclosure, several non-limiting features will now be discussed briefly.

In some aspects, the techniques described herein relate to a method including: receive training data including (i) a plurality of blood test results, (ii) a first label, and (iii) a second label, wherein each blood test result set, from the plurality of blood test results, includes a plurality of features, wherein each blood test result set, from the plurality of blood test results, includes a lipid panel test result, a metabolic panel test result, and a complete blood count test result, wherein each blood test result set, from the plurality of blood test results, is associated with a particular patient, and wherein each patient is associated with either (i) the first label indicating an identification of a disease or a condition or (ii) the second label indicating absence of the disease or the condition; training a machine learning model with the training data, wherein the machine learning model is configured to predict a likelihood that a patient has or will have the disease or the condition based at least in part on a new blood test result set for the patient; receiving a first blood test result set for a first patient; extracting a first plurality of features from the first blood test result set; applying the machine learning model to the first plurality of features as first input, wherein the machine learning model outputs a score that reflects a first likelihood that the first patient has or will have the disease or the condition; and outputting the score.

In some aspects, the techniques described herein relate to a method, wherein the disease or condition includes at least one of: preterm labor, Parkinson's disease, juvenile rheumatoid arthritis, Alzheimer's disease, sleep apnea, osteoporosis with or without pathological fracture, breast cancer, endometriosis, seropositive rheumatoid arthritis, seronegative rheumatoid arthritis, shingles, pulmonary hypertension, colon cancer, Crohn's disease, systemic lupus erythematosus, celiac disease, colitis, epilepsy, basal cell carcinoma, prostate cancer, Type 1 diabetes, Type 2 diabetes, prediabetes, autoimmune disease, cardiovascular disease, respiratory disease, asthma, chronic obstructive pulmonary disease, infectious disease, metabolic disorder, or neurodegenerative condition.

In some aspects, the techniques described herein relate to a method, wherein a fast plasma glucose feature is absent from the first plurality of features.

In some aspects, the techniques described herein relate to a method, further including: clustering patient-related data that results in a plurality of data clusters; and creating the training data based at least in part on a data cluster from the plurality of data clusters.

In some aspects, the techniques described herein relate to a method, wherein the training data further includes genetic marker data, further including: generating an additional feature for the patient corresponding to a genetic marker value, wherein the machine learning model receives the additional feature as second input.

In some aspects, the techniques described herein relate to a method, wherein the training data further includes ribonucleic acid sequence data, further including: generating an additional feature for the patient corresponding to a ribonucleic acid sequence value, wherein the machine learning model receives the additional feature as second input.

In some aspects, the techniques described herein relate to a method including: receiving training data including (i) a plurality of blood test results, (ii) a first label, and (iii) a second label, wherein each blood test result set, from the plurality of blood test results, includes a plurality of features, wherein each blood test result set, from the plurality of blood test results, includes a metabolic panel test result and a complete blood count test result, wherein each blood test result set, from the plurality of blood test results, is associated with a particular patient, and wherein each patient is associated with either (i) the first label indicating an identification of a disease or a condition or (ii) the second label indicating absence of the disease or the condition; training a machine learning model with the training data, wherein the machine learning model is configured to predict a likelihood that a patient has or will have the disease or the condition based at least in part on a new blood test result set for the patient; receiving a first blood test result set for a first patient; extracting a first plurality of features from the first blood test result set; applying the machine learning model to the first plurality of features as first input, wherein the machine learning model outputs a score that reflects a first likelihood that the first patient has or will have the disease or the condition; and outputting the score.

In some aspects, the techniques described herein relate to a method, wherein the disease or the condition includes preterm labor.

In some aspects, the techniques described herein relate to a method, wherein the training data further includes genetic marker data, further including: generating an additional feature for the patient corresponding to a genetic marker value, wherein the machine learning model receives the additional feature as second input.

In some aspects, the techniques described herein relate to a method, wherein the training data further includes ribonucleic acid sequence data, further including: generating an additional feature for the patient corresponding to a ribonucleic acid sequence value, wherein the machine learning model receives the additional feature as second input.

In some aspects, the techniques described herein relate to a method, wherein the machine learning model includes at least one of: a gradient-boosted tree, an ensemble model, a deep neural network, a transformer model, a reinforcement learning model, or a hybrid architecture combining a rule-based system and a machine learning system.

In some aspects, the techniques described herein relate to a method, further including: clustering patient-related data that results in a plurality of data clusters; and creating the training data based at least in part on a data cluster from the plurality of data clusters.

In some aspects, the techniques described herein relate to a method, wherein each data cluster of the plurality of data clusters corresponds to a particular disease or condition.

In some aspects, the techniques described herein relate to a system including: one or more data storage media configured to store specific computer-executable instructions; and one or more computer hardware processors configured to communicate with the one or more data storage media, wherein the specific computer-executable instructions are configured to cause the one or more computer hardware processors to at least: receive training data including (i) a plurality of blood test results, (ii) a first label, and (iii) a second label, wherein each blood test result set, from the plurality of blood test results, includes a plurality of features, wherein each blood test result set, from the plurality of blood test results, includes a metabolic panel test result and a complete blood count test result, wherein each blood test result set, from the plurality of blood test results, is associated with a particular patient, and wherein each patient is associated with either (i) the first label indicating an identification of a disease or a condition or (ii) the second label indicating absence of the disease or the condition; train a machine learning model with the training data, wherein the machine learning model is configured to predict a likelihood that a patient has or will have the disease or the condition based at least in part on a new blood test result set for the patient; receive a first blood test result set for a first patient; extract a first plurality of features from the first blood test result set; apply the machine learning model to the first plurality of features as first input, wherein the machine learning model outputs a score that reflects a first likelihood that the first patient has or will have the disease or the condition; and output the score.

In some aspects, the techniques described herein relate to a system, wherein a fast plasma glucose feature is absent from the first plurality of features.

In some aspects, the techniques described herein relate to a system, wherein the one or more computer hardware processors are configured to execute further computer-executable instructions to at least: cluster patient-related data that results in a plurality of data clusters; and create the training data based at least in part on a data cluster from the plurality of data clusters.

In some aspects, the techniques described herein relate to a system, wherein each data cluster of the plurality of data clusters corresponds to a particular disease or condition.

In some aspects, the techniques described herein relate to a system, wherein the training data further includes genetic marker data, wherein the one or more computer hardware processors are configured to execute further computer-executable instructions to at least: generate an additional feature for the patient corresponding to a genetic marker value, wherein the machine learning model receives the additional feature as second input.

In some aspects, the techniques described herein relate to a system, wherein the training data further includes ribonucleic acid sequence data, wherein the one or more computer hardware processors are configured to execute further computer-executable instructions to at least: generate an additional feature for the patient corresponding to a ribonucleic acid sequence value, wherein the machine learning model receives the additional feature as second input.

In some aspects, the techniques described herein relate to a system, wherein the machine learning model includes at least one of: a gradient-boosted tree, an ensemble model, a deep neural network, a transformer model, a reinforcement learning model, or a hybrid architecture combining a rule-based system and a machine learning system.

In some aspects, the techniques described herein relate to a method for diagnosing diabetes including: receiving a test result set for a patient; generating, from at least the test result set, a plurality of features including: a first feature corresponding to a total cholesterol value, a second feature corresponding to a high density cholesterol value, a third feature corresponding to a triglyceride value, a fourth feature corresponding to a hemoglobin value, a fifth feature corresponding to a white blood cell count value, a sixth feature corresponding to a red blood cell count value, a seventh feature corresponding to a platelet count value, an eighth feature corresponding to a glucose value, a ninth feature corresponding to a creatinine value, a tenth feature corresponding to a calcium value, an eleventh feature corresponding to an albumin value, a twelfth feature corresponding to a sodium value, a thirteenth feature corresponding to a protein value, a fourteenth feature corresponding to a potassium value, a fifteenth feature corresponding to an alanine aminotransferase value, a sixteenth feature corresponding to a bicarbonate value, a seventeenth feature corresponding to an aspartate aminotransferase value, an eighteenth feature corresponding to a chloride value, a nineteenth feature corresponding to an alkaline phosphatase value, a twentieth feature corresponding to a blood urea nitrogen value, a twenty-first feature corresponding to a bilirubin value, a twenty-second feature corresponding to an age value, a twenty-third feature corresponding to a gender value, and a twenty-fourth feature corresponding to a smoker value; applying a trained machine learning model to the plurality of features as input, wherein the trained machine learning model outputs a score that reflects a likelihood that the patient has diabetes; and outputting the score.

In some aspects, the techniques described herein relate to a method, wherein at least one of the third feature, the fifth feature, the sixth feature, the ninth feature, the tenth feature, the thirteenth feature, the fifteenth feature, the seventeenth feature, the eighteenth feature, the nineteenth feature, or the twentieth feature has a null value.

In some aspects, the techniques described herein relate to a method for diagnosing diabetes including: receiving a test result set for a patient; generating, from at least the test result set, a plurality of features including: a first feature corresponding to a total cholesterol value, a second feature corresponding to a high density cholesterol value, a third feature corresponding to a hemoglobin value, a fourth feature corresponding to a glucose value, a fifth feature corresponding to an albumin value, a sixth feature corresponding to a sodium value, a seventh feature corresponding to a potassium value, an eighth feature corresponding to a bicarbonate value, a ninth feature corresponding to a bilirubin value, a tenth feature corresponding to an age value, an eleventh feature corresponding to a gender value, and a twelfth feature corresponding to a smoker value; applying a trained machine learning model to the plurality of features as input, wherein the trained machine learning model outputs a score that reflects a likelihood that the patient has diabetes; and outputting the score.

In some aspects, the techniques described herein relate to a method for diagnosing prediabetes including: receiving a test result set for a patient; generating, from at least the test result set, a plurality of features including: a first feature corresponding to a total cholesterol value, a second feature corresponding to a low density cholesterol value, a third feature corresponding to a triglyceride value, a fourth feature corresponding to a hemoglobin value, a fifth feature corresponding to a hematocrit value, a sixth feature corresponding to a white blood cell count value, a seventh feature corresponding to a red blood cell count value, an eighth feature corresponding to a platelet count value, a ninth feature corresponding to a glucose value, a tenth feature corresponding to a creatinine value, an eleventh feature corresponding to a calcium value, a twelfth feature corresponding to an albumin value, a thirteenth feature corresponding to a sodium value, a fourteenth feature corresponding to a protein value, a fifteenth feature corresponding to a potassium value, a sixteenth feature corresponding to an alanine aminotransferase value, a seventeenth feature corresponding to a bicarbonate value, an eighteenth feature corresponding to an aspartate aminotransferase value, a nineteenth feature corresponding to a chloride value, a twentieth feature corresponding to an alkaline phosphatase value, a twenty-first feature corresponding to a blood urea nitrogen value, a twenty-second feature corresponding to a bilirubin value, a twenty-third feature corresponding to an age value, a twenty-fourth feature corresponding to a gender value, and a twenty-fifth feature corresponding to a smoker value; applying a trained machine learning model to the plurality of features as input, wherein the trained machine learning model outputs a score that reflects a likelihood that the patient has prediabetes; and outputting the score.

In some aspects, the techniques described herein relate to a method, wherein at least one of the second feature, the third feature, the fourth feature, the fifth feature, the sixth feature, the eighth feature, the tenth feature, the thirteenth feature, the fourteenth feature, the fifteenth feature, the sixteenth feature, the eighteenth feature, the nineteenth feature, the twenty-first feature, the twenty-second feature, the twenty-fourth feature corresponding to a gender value, or the twenty-fifth feature has a null value.

In some aspects, the techniques described herein relate to a method for diagnosing prediabetes including: receiving a test result set for a patient; generating, from at least the test result set, a plurality of features including: a first feature corresponding to a total cholesterol value, a second feature corresponding to a hemoglobin value, a third feature corresponding to a red blood cell count value, a fourth feature corresponding to a glucose value, a fifth feature corresponding to a calcium value, a sixth feature corresponding to an albumin value, a seventh feature corresponding to a bicarbonate value, an eighth feature corresponding to an alkaline phosphatase value, and a ninth feature corresponding to an age value; applying a trained machine learning model to the plurality of features as input, wherein the trained machine learning model outputs a score that reflects a likelihood that the patient has prediabetes; and outputting the score.

In some aspects, the techniques described herein relate to a method, wherein a fast plasma glucose feature is absent from the plurality of features.

In some aspects, the techniques described herein relate to a method for predicting risk of preterm labor including: receiving a blood test result set for a patient; generating, from at least the blood test result set, a plurality of features including: a first feature corresponding to a hemoglobin value, a second feature corresponding to a white blood cell count value, a third feature corresponding to a platelet count value, a fourth feature corresponding to an albumin value, a fifth feature corresponding to a sodium value, a sixth feature corresponding to a protein value, a seventh feature corresponding to a bicarbonate value, an eighth feature corresponding to an aspartate aminotransferase value, a ninth feature corresponding to a chloride value, a tenth feature corresponding to an alkaline phosphatase value, an eleventh feature corresponding to a blood urea nitrogen value, a twelfth feature corresponding to a bilirubin value, a thirteenth feature corresponding to an age value, and a fourteenth feature corresponding to a smoker value; applying a trained machine learning model to the plurality of features as input, wherein the trained machine learning model outputs a score that reflects a likelihood that the patient will have preterm labor; and outputting the score.

In some aspects, the techniques described herein relate to a method, wherein the trained machine learning model at least one of: a gradient-boosted tree, an ensemble model, a deep neural network, a transformer model, a reinforcement learning model, or a hybrid architecture combining a rule-based system and a machine learning system.

In some aspects, the techniques described herein relate to a method including: collecting patient-related data, applying one or more transformations to the patient-related data, creating a training set including the modified patient-related data, and training the machine learning model (such as a neural network) using the training set.

In various aspects, systems and/or computer systems are disclosed that comprise a computer readable storage medium having program instructions embodied therewith, and one or more processors configured to execute the program instructions to cause the one or more processors to perform operations comprising one or more of the above- and/or below-aspects (including one or more aspects of the appended claims).

In various aspects, computer-implemented methods are disclosed in which, by one or more processors executing program instructions, one or more of the above- and/or below-described aspects (including one or more aspects of the appended claims) are implemented and/or performed.

### BRIEF DESCRIPTION OF THE DRAWINGS

These and other features, aspects, and advantages are described below with reference to the drawings, which are intended for illustrative purposes and should in no way be interpreted as limiting the scope of the embodiments. Furthermore, various features of different disclosed embodiments can be combined to form additional embodiments, which are part of this disclosure. In the drawings, like reference characters can denote corresponding features throughout similar embodiments. The following is a brief description of each of the drawings.

FIG. 1 is a block diagram depicting a workflow for machine learning based disease or condition prediction.

FIG. 2 is a block diagram depicting an illustrative network environment for implementing a machine learning prediction system.

FIGS. 3A, 3B, 3C, and 3D depict non-limiting test results and/or features.

FIG. 4 depicts non-limiting diseases and conditions associated with patients and laboratory tests.

FIG. 5 depicts a workflow illustrating a non-limiting novel clinical insight.

FIG. 6 is a flow chart depicting a method for machine learning based disease or condition predictions.

FIG. 7 depicts the test results of diabetes and control cohorts.

FIGS. 8A-8B depict the results of automated feature selection for diabetes diagnosis ranked by accuracy.

FIG. 9 depicts the results of automated feature selection for preterm labor prediction ranked by accuracy.

FIG. 10 is a block diagram illustrating an example computing system with which various methods and systems discussed herein may be implemented.

### DETAILED DESCRIPTION

Current healthcare tests and the ability to predict when someone may get a disease are limited. Many healthcare tests are acute to the diagnosis of particular diseases. In other words, a patient may be well into having a disease when existing healthcare tests indicate a positive result. As described above, many existing healthcare tests look for a specific indicator (such as the A1C test that measures red blood cells that have glucose-coated hemoglobin) and a clinician can use the test results to make a specific diagnosis. However, a clinician or patient may order a condition or disease specific test when the patient has had the condition or disease for a while. It would be advantageous if conditions or diseases could be detected much earlier based on routine healthcare test results (such as routine blood test results).

Generally described, aspects of the present disclosure are directed to systems and methods for machine learning based disease or condition prediction from healthcare test results either at a single time point or across multiple time points. The system can be configured to estimate disease probability, stratify patients by risk level, and/or generate risk trajectories over time. The framework can be designed to be extensible to future disease categories, novel biomarkers, and/or evolving machine learning models. The methodology applies to any disease or condition where blood-based, genetic, imaging, environmental, or real-time physiological data provide diagnostic insights. This includes known and yet-to-be-discovered conditions that can benefit from machine learning-driven clinical risk assessment. Advantageously, the systems and methods described herein can make predictions based on routine healthcare test results. The systems and methods described herein can predict the presence or absence of a disease or condition, which can include screening, i.e., early disease or condition detection. Routine healthcare test results can include a number of typical clinical measures (such as forty, fifty, sixty measures) each with a separate value, which may be accompanied with a “typical” range for each value. However, important aspects of some of the systems and methods described herein relate to the key observation that the “typical” ranges for clinical measures can be pretty wide and being within a “typical” range can include patients that are both normal and abnormal, i.e., without a particular condition and with a particular condition, for example. Moreover, diagnosing a disease or condition on a single test basis with existing healthcare tests may not be refined. Unlike a clinician, where it may be impossible for a single person to consider many clinical dimensions simultaneously (such as forty, fifty, sixty measures), machine learning can be used to make predictions based on multiple features from healthcare tests that improve on existing healthcare diagnostic/screening methods. Machine learning algorithms can be used to detect patterns within the data. The systems and methods described herein can be used to make predictions regarding diseases or conditions, such as, but not limited to, preterm labor, Parkinson's disease, juvenile rheumatoid arthritis, Alzheimer's disease, sleep apnea, osteoporosis with or without pathological fracture, breast cancer, endometriosis, seropositive rheumatoid arthritis, seronegative rheumatoid arthritis, shingles, pulmonary hypertension, colorectal cancer, Clostridioides difficile (C. Diff), Crohn's disease, systemic lupus erythematosus, celiac disease, colitis, epilepsy, basal cell carcinoma, prostate cancer, Metabolic Associated Fatty Liver disease (MAFLD), Iron Deficiency Anemia, Elevated C-Reactive Protein (CRP), Type 1 diabetes, Type 2 diabetes, prediabetes, autoimmune diseases, cardiovascular diseases, respiratory diseases (including asthma and chronic obstructive pulmonary disease), infectious diseases, metabolic disorders, neurodegenerative conditions, and other disease/conditions, such as any other disease/condition for which blood-based biomarkers provide predictive value.

Generally described, aspects of the present disclosure are directed to systems and methods for improved disease or condition prediction. Existing healthcare tests look for a specific indicator (such as the A1C test that measures red blood cells that have glucose-coated hemoglobin) and a clinician can use the test results to make a specific diagnosis. Advantageously, the systems and methods described herein can make predictions based on routine healthcare test results. The systems and methods described herein can predict the presence or absence of a disease or condition, which can include screening, i.e., early disease or condition detection. Moreover, the systems and methods described herein can utilize machine learning and training to improve over the accuracy of existing tests. A method of training a machine learning model (such as a neural network) for disease or condition prediction described herein can include: collecting patient-related data, applying one or more transformations to the patient-related data, creating a training set including the modified patient-related data, and training the machine learning model (such as the neural network) using the training set. Moreover, the approaches described herein can include some pre-processing steps that reduce the computational complexity of machine learning training. Accordingly, some of the systems and methods described herein can be more computationally efficient than some of existing methods for machine learning training and therefore improve the operation of a computer. Accordingly, the systems and methods described herein can improve disease or condition prediction technology and/or machine learning technology.

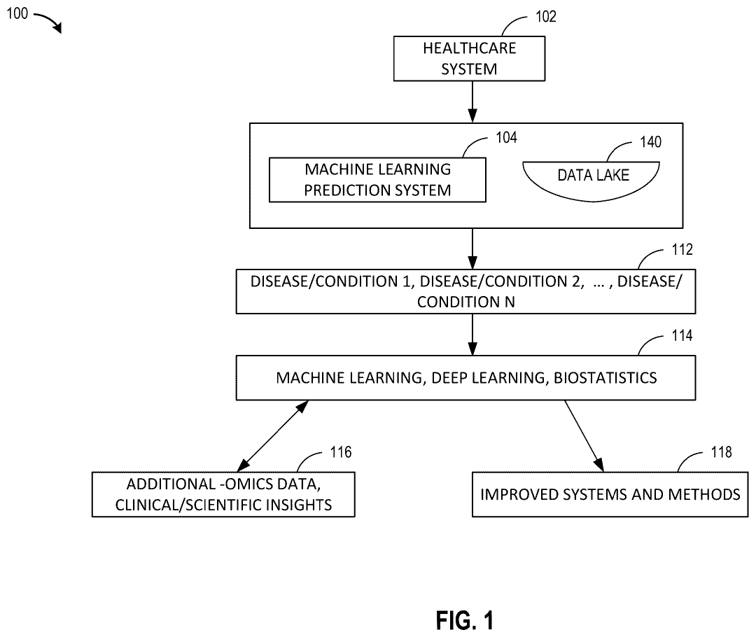

Turning to FIG. 1, a block diagram is shown depicting a workflow 100 for machine learning based disease or condition prediction. The workflow 100 can leverage data from a healthcare system and machine learning in improved systems and methods for novel diagnostic/prognostic tools, novel clinical insights, novel care process models, and/or novel therapeutic targets. The workflow 100 and machine learning prediction system 104 can be applied to any test result, such as, but not limited to routine blood test results.

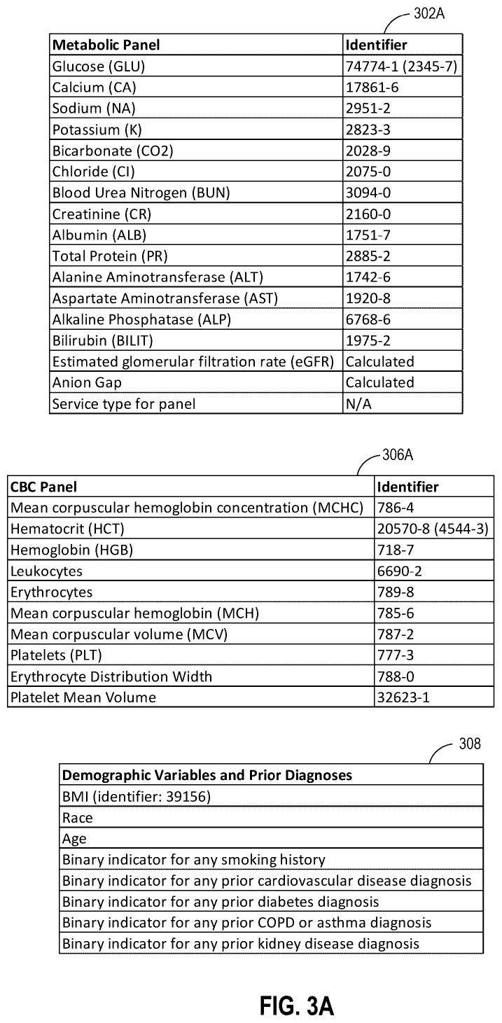

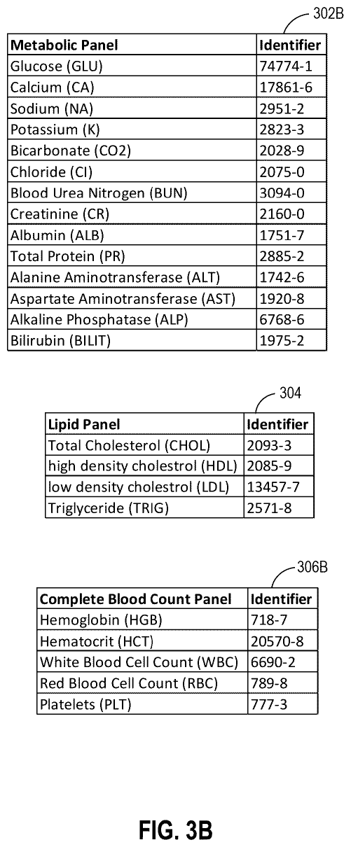

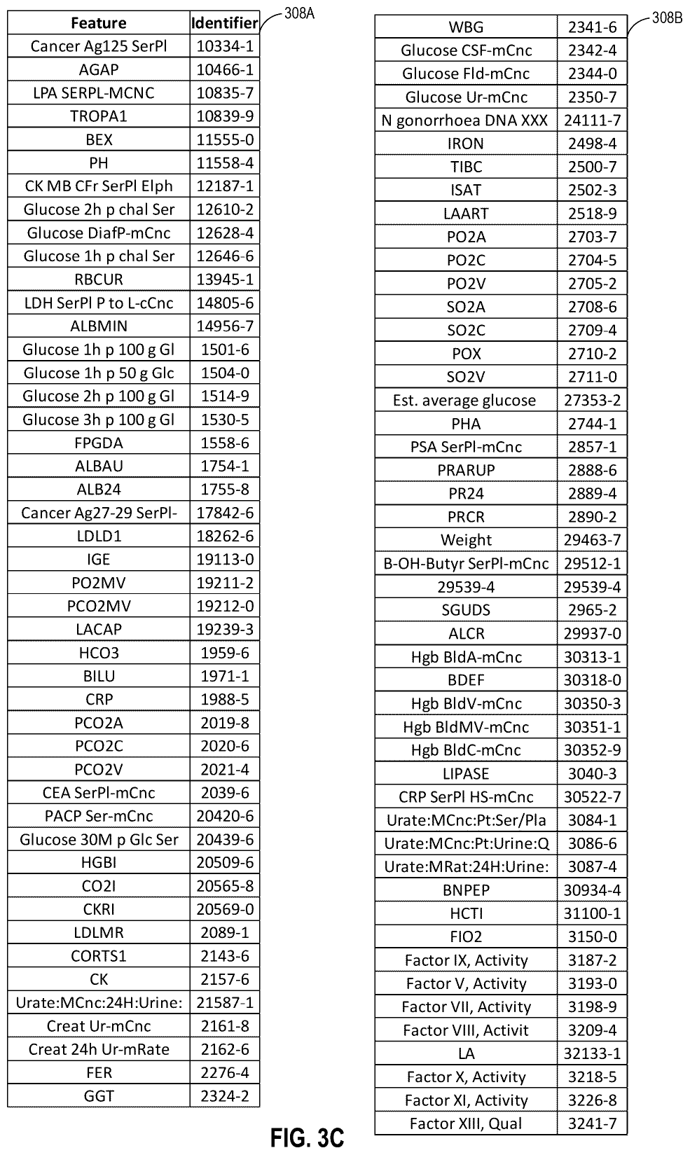

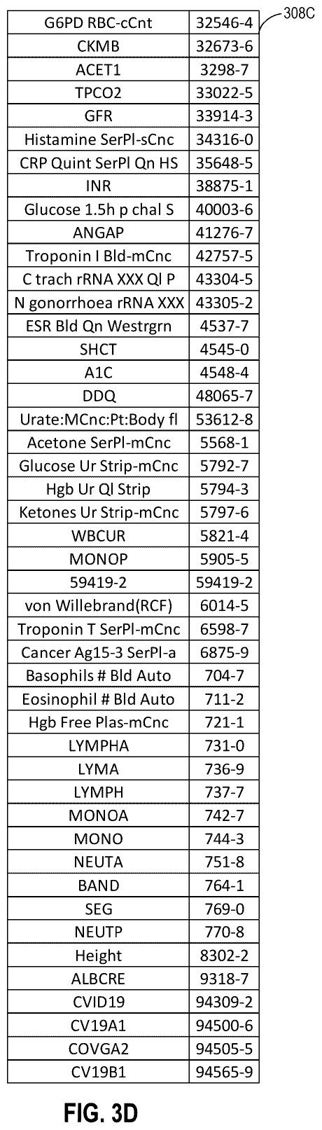

A machine learning prediction system 104 and a data lake 140 can receive data from a healthcare system 102, which can be received on an irregular or regular basis with updates. The data from the healthcare system 102 can include, but is not limited to, de-identified historical data and/or electronic heath records. The electronic health records can include, but are not limited to, laboratory tests, slide images, radiology images, unstructured notes, genomics data (such as DNA related data), transcriptomics data (such as RNA related data), proteomics data (such as proteins related data), phenomics data (such as phenotypes data), and/or metabolomics data (such as metabolites data). The data from the healthcare system 102 can be stored in the data lake 140. In some embodiments, the received data and/or the data lake 140 can include millions of longitudinal patient journeys (such as, but not limited to, one or more blood test result sets for a patient from a single time point or multiple time points); linked bio-specimens; trillions of genetic variants; hundreds of thousands of new annual cases; digital images; and/or radiology images. Additional details regarding laboratory tests/features are described herein, such as with respect to FIGS. 3A, 3B, 3C, and 3D. The machine learning prediction system 104 can process the test results to extract longitudinal trends in biomarker levels over time; determine, from at least the blood test results, features, including temporal features derived from historical test values; and apply a trained machine learning model to predict disease likelihood or risk score. The data lake 140 can include information indicating performed medical services and procedures furnished by physicians and other health care professionals (such as CPT-4 codes), medications, prescriptions, and/or allergies, which can be used for machine learning training.

As described herein, the machine learning prediction system 104 can predict the presence or absence of a disease or condition based on healthcare-related data. Healthcare-related data can include, but is not limited to, routine blood test results, imaging data (such as X-rays, MRIs, CT scans), real-time/near-time continuous health monitoring from wearable devices, environmental exposure data (such pollution-related data or allergens-related data), genetic and/or transcriptomic data, and/or data from other healthcare-related data sources.

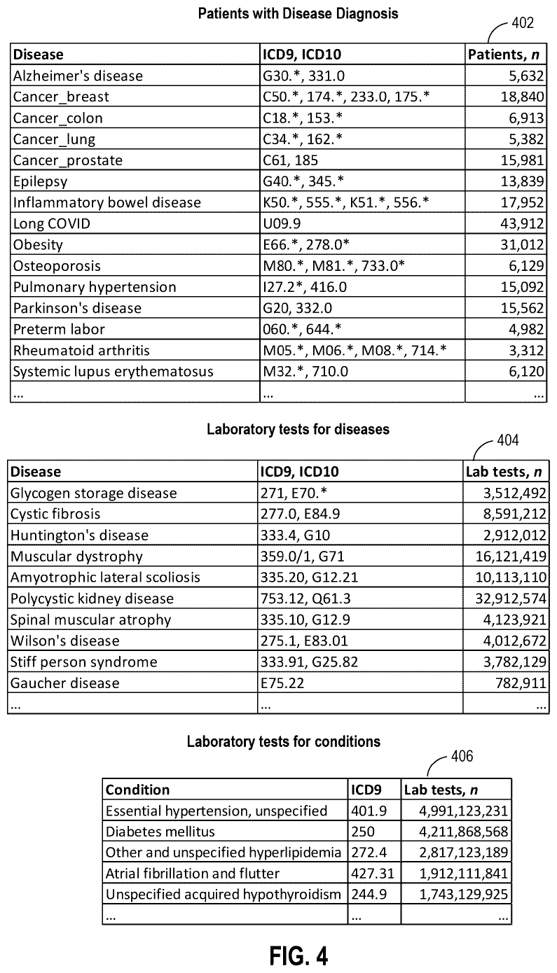

In particular, the machine learning prediction system 104 can, with the data from the data lake 140, determine one or more diseases/conditions 112. The machine learning prediction system 104 can query the data from the data lake 140 to determine the diseases/conditions 112. For example, the machine learning prediction system 104 can query diagnoses codes and/or diagnostic tests to determine at least some training data. Additional details regarding querying diagnoses codes and/or diagnostic tests are described herein, such as with respect to FIG. 4. In some embodiments, after determination of the diseases/conditions 112, the machine learning prediction system 104 can perform data cleaning and/or preprocessing on the training data. A disease or condition predicted by the machine learning prediction system 104 (such as a risk category or a categorization) can include, but is not limited to, Acute lymphocytic leukemia, Acute myeloid leukemia, Advanced NSCLC, Alopecia areata, Alzheimer's disease, Amyotrophic lateral sclerosis (ALS), Ankylosing spondylitis, Anxiety disorders, Arthropathic psoriasis (Psoriatic arthritis), Asthma, Asthma mild intermittent, Asthma_mild persistent, Asthma_moderate persistent, Asthma_severe persistent, Atherosclerosis, Atherosclerosis with elevated Lp (a), Atherosclerosis with PAD, Atopic dermatitis, Basal cell carcinoma, Bipolar disorder, Bladder cancer, Breast cancer, Bronchitis, Cardiac amyloidosis, Cardiomyopathy, Celiac disease, Cerebal cavernous malformation*, Cervical cancer, Chronic cough, Chronic hepatitis B (CHB), Chronic kidney disease (CKD), Chronic lymphocytic leukemia, Chronic myeloid leukemia, Chronic rhinosinusitis with nasal polyps, Chronic spontaneous urticaria, Clostridioides difficile Infection, Colon cancer, Colorectal cancer, COPD, Coronary artery disease, Crohn's disease, Cutaneous lupus erythematosus, Deep vein thrombosis, Depressive disorders, recurrent, Depressive disorders, single episode, Dermatomyositis, Diabetes, Discoid lupus crythematosus, Duchenne muscular dystrophy (DMD), Endometrial cancer, Endometriosis, Eosinophilic (Type 2) asthma, Eosinophilic esophagitis, Epilepsy, Familial adenomatous polyposis, Focal segmental glomerulosclerosis, Frontotemporal dementia, Gastric and esophageal, Giant cell arteritis (GCA), Glioblastoma, Gonococcal infection, Gout, Granulomatosis with polyangiitis (GPA), Graves' disease, Growth hormone deficiency, Head and neck cancer, Heart failure, Hemophilia, A and B, Hepatocellular carcinoma (HCC), Herpes simplex virus (HSV), HFpEF, Hidradenitis suppurativa, HIV, Huntington's disease (HD), Hypertension, Hyperthryoidism, Hypophosphatasia*, Hypothryoidism, IBD, Idiopathic pulmonary fibrosis, Idiopathic thrombocytopenia purpura (ITP), IgG4 related disease, Inclusion body myositis (IBM), Infertility (M/F), Influenza, Iron-deficiency anemia, Kidney cancer, Klebsiella infection, Liver and bile duct cancer, Long COVID, Lower back pain, Lung cancer, Lung cancer with mesothelioma, Lung fibrosis, Lupus nephritis, Melanoma, Meningitis, Merkel cell carcinoma, Mesotheloima, Metastatic CRC, Mixed connective tissue disease, Multiple myeloma, Multiple sclerosis, Multiple system atrophy, Myasthenia gravis (MG), Myelodysplastic syndrome (MDS), Myelofibrosis, Myopathy, unspecified, Myotonic muscular dystrophy, type 1 and 2 (DM1, DM2), Neurofibromatous type 2, Neuromyelitis optica spectrum disorder, Non-Hodgkin lymphoma, Non-infectious uveitis, Nonalcoholic steatohepatitis (NASH)/Metabolic dysfunction-associated steatohepatitis (MASH), Obesity, Oral cavity and pharynx cancer, Osteoarthritis, Osteoporosis, Ovarian cancer, Pancreatic cancer, Paratyphoid fever, PD, Pertussis (whooping cough), Pneumonia (pneumococcal), Polyarteritis nodosa (PAN), Polycythemia vera, Polymyositis, Pompe disease, Pre-diabetes, Pre-eclampsia, Preterm labor, Prostate cancer, Prurlgo nodaris, Psoriasis, Pulmonary hypertension, Respiratory syncytial virus (RSV), Rheumatoid arthritis (RA)*, Salmonella infection, Sarcoidosis, Schizophrenia, Sepsis (pneumococcal), Shigellosis (Shigella infection), Shingles, Sickle cell disorders, Sjogren's syndrome, Soft tissue sarcoma, Stroke, Systemic lupus erythematosus (SLE), Systemic sclerosis, Systemic sclerosis with lung involvement*, Thalassemia, Thyroid cancer, Thyroid eye disease (TED), Traumatic brain injuries (TBI), Typhoid fever, Ulcerative colitis, Urea cycle disorder, Urinary tract infections (UTI), and/or Von Willebrand disease.

At block 114, the machine learning prediction system 104 can perform machine learning (such as, but not limited to, supervised machine learning, unsupervised machine learning, and/or reinforcement machine learning), deep learning, and/or biostatistics that results in improved systems and methods 118. As described herein, the improved systems and methods 118 can be determined where a threshold is satisfied. For example, the improved systems and methods 118 can satisfy a sensitivity and/or specificity threshold (such as greater than or equal to 85%) and/or a statistical significance threshold (such as a p-value). If a threshold is not met, then additional data can be used in machine learning, deep learning, and/or biostatistics. The machine learning, deep learning, and/or biostatistics at block 116 can use additional data, such as, but not limited to, genomics data, transcriptomics data, proteomics data, phenomics data, metabolomics data, and/or clinical/scientific insights.

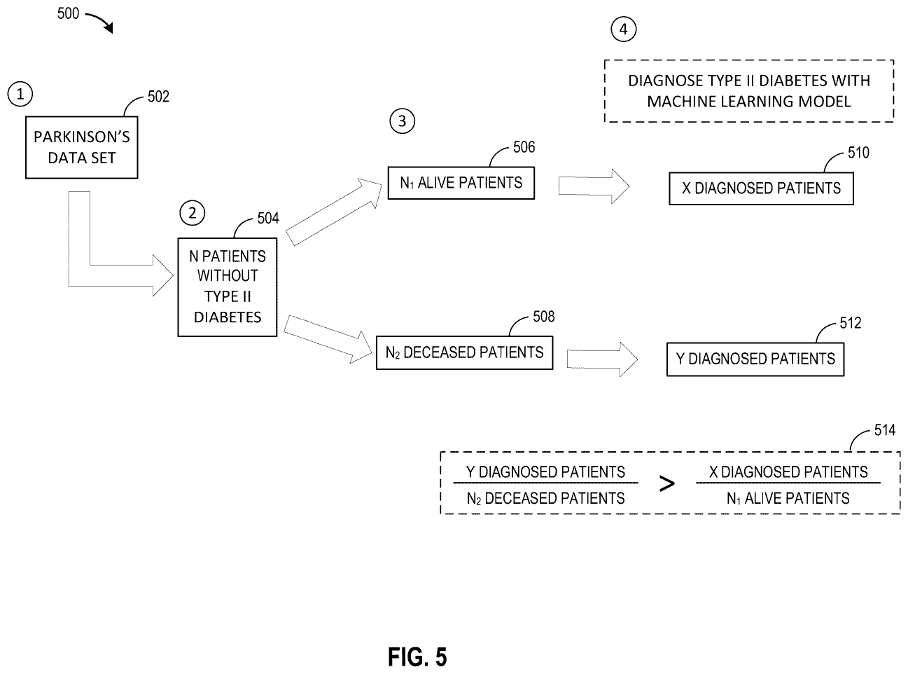

As described herein, the improved systems and methods 118 can include or be related to novel screening/diagnostic/prognostic tools, novel clinical insights, novel care process models, and/or novel therapeutic targets. As described herein, the developed novel screening/diagnostic/prognostic tools can be directed towards diseases/conditions, such as, but not limited to, diabetes, prediabetes, preterm labor, other cohorts, etc. The developed novel screening/diagnostic/prognostic tools can have different levels of accuracy (such as area under the receiver operating characteristic curve (AUC), sensitivity, and/or specificity). As described herein, the diseases/conditions can include, but are not limited to, preterm labor, Parkinson's disease, juvenile rheumatoid arthritis, Alzheimer's disease, sleep apnea, osteoporosis with or without pathological fracture, breast cancer, endometriosis, seropositive rheumatoid arthritis, seronegative rheumatoid arthritis, shingles, pulmonary hypertension, colorectal cancer, Clostridioides difficile (C. Diff), Crohn's disease, systemic lupus erythematosus, celiac disease, colitis, epilepsy, basal cell carcinoma, prostate cancer, Metabolic Associated Fatty Liver disease (MAFLD), Iron Deficiency Anemia, Elevated C-Reactive Protein (CRP), Type 1 diabetes, Type 2 diabetes, prediabetes, autoimmune diseases, cardiovascular diseases, respiratory diseases (including asthma and chronic obstructive pulmonary disease), infectious diseases, metabolic disorders, neurodegenerative conditions, and other disease/condition, such as any other disease/condition for which blood-based biomarkers provide predictive value. The machine learning prediction system 104 can provide patient assistance based on novel clinical insights and/or care process models. For example, the machine learning prediction system 104 can diagnose previously undiagnosed diabetes in Parkinson's disease patients and provide a warning that such patients may be at increased risk for mortality. Additional details regarding novel clinical insights and/or care process models are described herein, such as with respect to FIG. 5.

The machine learning prediction system 104 can determine novel therapeutic targets, such as, but not limited to identification of novel pathogenic gene variants and/or chemoinformatics. The data lake 140 can include genome sequencing data, such as whole genome sequencing data. The machine learning prediction system 104 can determine novel variants in genes that could be involved in different diseases or conditions, such as, diabetes or cancer. The novel variants in genes may have not been previously categorized as pathogenic. The machine learning prediction system 104 can include a pathogenicity predictor that predicts variants as either benign or pathogenic. The output from the pathogenicity predictor can be used to prioritize which variants can be studied for function characterization. The functional characterization can identify novel variants that have negative effects, which can be used for therapeutic targets. To train the pathogenicity predictor, the inputs include patients with or without the particular variant and labels for patients with and without a disease or condition.

In some embodiments, the machine learning prediction system 104 can, for each disease cohort, process results for individuals with laboratory results and diagnosis codes recorded between a time period (such as ten years of results). In some cases, different diseases can use different time periods; for example, for cancers, a different time period for laboratory results and diagnosis codes (such as fifteen years of results). Predictive features can include, but are not limited to, a complete blood count panel, a metabolic panel, and/or other features (such as Body Mass Index (BMI) and/or smoking status). The predictive features can be recent, such as being taken within a year prior to or on the date of initial diagnosis. Example features are described herein, such as with respect to FIGS. 3A, 3B, 3C, and 3D. The machine learning prediction system 104 can incorporate prior diagnoses of risk factors across several disease areas, such as, but not limited to, cardiovascular disease, kidney disease, diabetes, and/or COPD/asthma. For the control group of each disease, individuals with any history of the disease of interest can be excluded from the training data. To ensure comparable data, a time period (such as a month) during which each control group individual had the highest number of labs performed can be determined and the lab values from that month can be used for training purposes. In some cases, smoking status for the control group can be assigned based on a same time period (such as year) the lab values were obtained. Prior diagnoses of risk factors can be defined as occurring before the lab result dates.

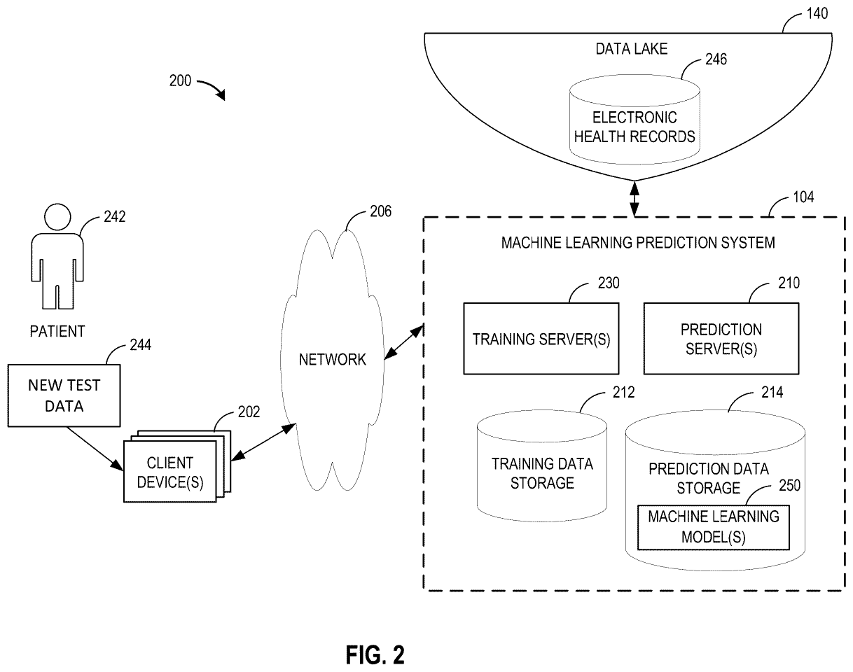

Turning to FIG. 2, an illustrative network environment 200 is shown in which a machine learning prediction system 104 may make disease or condition predictions. The network environment 200 may include one or more client devices 202, a data lake 140, and the machine learning prediction system 104. The constituents of the network environment 200 may be in communication with each other either locally or over a network 206. While certain constituents of the network environment 200 are depicted as being in communication with one another, any constituent of the network environment 200 can communicate with any other constituent of the network environment 200; however, not all of these communication lines are depicted in FIG. 2.

The data lake 140 can store electronic health records 246. The data lake 140 and/or the machine learning prediction system 104 may not store any personal identifiable information (PII) and may be compliant with one or more regulations, such as Health Insurance Portability and Accountability Act (HIPAA). Example electronic health records 246 can include, but are not limited to, test results from a lipid panel, a metabolic test (such as a comprehensive metabolic panel (CMP)), and/or complete blood count (CBC) test. Additional electronic health records 246 can include, but are not limited to, diagnoses or records that indicate particular diseases or conditions for particular patients (such as, but not limited to, T2D diagnosis, a prediabetes diagnosis, or preterm labor, etc.). Additional types of diseases or conditions, which can be stored in the data lake 140, are described herein. Additional electronic health records 246 can include, but are not limited to, proteomics data, metabolomics data, epigenomics data, genetic data (such as one or more genetic markers), and/or ribonucleic acid (RNA) data. The electronic health records 246 can include demographics data, such as, but not limited to, gender, age, or whether the patient was/is a smoker. The electronic health records 246 can be queried by patient without the use of any PII.

The machine learning prediction system 104 may include one or more training servers 230, one or more prediction servers 210, a training data storage 212, and a prediction data storage 214. The training server 230 can generate training data from the electronic health records 246 from the data lake 140. The training server 230 or a data analyst can query the electronic health records 246 by patient and/or other criteria. For example, patients that have particular test results, such as those patients with blood test(s) such as a lipid panel, a metabolic test, and/or a CBC test, and have been detected or identified as having or not having a particular disease or condition (such as, but not limited to, diabetes, prediabetes, or preterm labor) can be identified from the data lake 140. Example training data can include blood test results and labels. Each blood test result set can include multiple features. Each blood test result set can include a lipid panel test result, a metabolic panel test result, and/or a complete blood count test result. As described herein, each blood test result set can be associated with a particular patient. Each patient can be associated with either a first label indicating an identification of a disease or condition (such as, but not limited to, diabetes, prediabetes, or preterm labor) or a second label indicating an absence of the disease or condition. The training data can be stored in the training data storage 212.

The training server 230 can train one or more machine learning models 250 using the training data. The training server 230 can train the one or more machine learning models 250 with supervised machine learning algorithms, which can use the labelled training data. The one or more machine learning models 250 can be trained to receive input data and output a score that reflects a likelihood that a patient has or will have the disease or condition. The score can indicate a probability, risk category, and/or a classification. For example, the score(s) can indicate a likelihood of the patient being associated with a classification. The one or more machine learning models 250 can make inferences. Each of the one or more machine learning models 250 can be trained to predict different diseases or conditions. The prediction server 210 can make a prediction based on input data (such as but not limited to the new test data 244) and the one or more trained machine learning models 250. Advantageously, as described herein, the new input data may be missing some feature data (such as having one or more null values where a patient does not have particular test results) and the trained machine learning model can still provide an accurate prediction even with the missing data. The machine learning prediction system 104 can provide the prediction to the client devices 202. In some embodiments, the prediction result(s) can be presented in a graphical user interface. For example, a user computing device can access the graphical user interface. The graphical user interface can display outcome predictions.

In some embodiments, the client device 202 can be a server from a healthcare organization, such as a healthcare provider and/or a clinical laboratory organization. Additionally or alternatively, the client device 202 can include, but is not limited to, a laptop or tablet computer, personal computer, personal digital assistant (PDA), hybrid PDA/mobile phone, smart wearable device (such as a smart watch), mobile phone, and/or a smartphone. A patient 242 can take a healthcare test (such as a routine blood test), which can result in the new test data 244. The client device 202 can provide the new test data 244 to the machine learning prediction system 104.

The data lake 140, the training data storage 212, and/or the prediction data storage 214 may be embodied in hard disk drives, solid state memories, any other type of non-transitory computer-readable storage medium. The data lake 140, the training data storage 212, and/or the prediction data storage 214 may also be distributed or partitioned across multiple local and/or remote storage devices. The data lake 140, the training data storage 212, and/or the prediction data storage 214 may include a data store. As used herein, a “data store” can refer to any data structure (and/or combinations of multiple data structures) for storing and/or organizing data, including, but not limited to, relational databases (e.g., Oracle databases, MySQL databases, etc.), non-relational databases (e.g., NoSQL databases, etc.), key-value databases, in-memory databases, tables in a database, and/or any other widely used or proprietary format for data storage.

The network 206 may be any wired network, wireless network, or combination thereof. In addition, the network 206 may be a personal area network, local area network, wide area network, cable network, satellite network, cellular telephone network, or combination thereof. In addition, the network 206 may be a publicly accessible network of linked networks, possibly operated by various distinct parties, such as the Internet. In some embodiments, the network 206 may be a private or semi-private network, such as a corporate or university intranet. The network 206 may include one or more wireless networks, such as a Global System for Mobile Communications (GSM) network, a Code Division Multiple Access (CDMA) network, a Long-Term Evolution (LTE) network, or any other type of wireless network. The network 206 can use protocols and components for communicating via the Internet or any of the other aforementioned types of networks, such as HTTP, TCP/IP, and/or UDP/IP.

The client devices 202 and/or the machine learning prediction system 104 may each be embodied in a plurality of devices. Each of the client devices 202 and/or the machine learning prediction system 104 may include a network interface, memory, hardware processor, and non-transitory computer-readable medium drive, all of which may communicate with each other by way of a communication bus. The network interface may provide connectivity over the network 206 and/or other networks or computer systems. The hardware processor may communicate to and from memory containing program instructions that the hardware processor executes in order to operate the client devices 202 and/or the machine learning prediction system 104. The memory generally includes RAM, ROM, and/or other persistent and/or auxiliary non-transitory computer-readable storage media.

Additionally, in some embodiments, the machine learning prediction system 104 or components thereof (such as the training server 230, the prediction server 210, the training data storage 212, and/or the prediction data storage 214) and/or the data lake 140 are implemented by one or more virtual machines implemented in a hosted computing environment. The hosted computing environment may include one or more rapidly provisioned and/or released computing resources. The computing resources may include hardware computing, networking and/or storage devices configured with specifically configured computer-executable instructions. A hosted computing environment may also be referred to as a “serverless,” “cloud,” or distributed computing environment.

FIG. 3A depicts non-limiting test results and/or features in a metabolic panel table 302A, a complete blood count panel table 306A, and a demographic variables and prior diagnoses table 308. These features and any of the features described herein can be derived from other sources, such as a bodily sample (such as a hair, tissue, urine, or saliva sample). Moreover, these features and any of the features described herein can encompass a co-regulated/associated marker/biomarker. For example, a cholesterol feature can include some other marker/biomarker that is associated with a cholesterol value for a patient. As described herein, an advantage to making predictions based on metabolic panel, a complete blood count panel, and demographic variables is that they can be routinely available for many patients. As shown in the metabolic panel table 302A, metabolic blood test results and/or features can include, but are not limited to, a glucose (GLU), calcium (CA), sodium (NA), potassium (K), bicarbonate (CO2), chloride (CI), blood urea nitrogen (BUN), creatinine (CR), albumin (ALB), total protein (PR), alanine aminotransferase (ALT), aspartate aminotransferase (AST), alkaline phosphatase (ALP), bilirubin (BILIT), an estimated glomerular filtration rate (eGFR), and/or an anion gap test result and/or feature. The service type for the metabolic panel can be utilized as a feature. As shown in the complete blood count panel table 306A, complete blood count test results and/or features can include, but are not limited to, a mean corpuscular hemoglobin concentration (MCHC), a hematocrit (HCT), hemoglobin (HGB), leukocytes (white blood cell count), erythrocytes (red blood cell count), mean corpuscular hemoglobin (MCH), mean corpuscular volume (MCV), platelets (PLT), erythrocyte distribution width, and/or platelet mean volume test result and/or feature. As shown in the demographic variables and prior diagnoses table 308, demographic variables and prior diagnoses test results and/or features can include a body mass index (BMI), race, age, binary indicator for any smoking history, binary indicator for any prior cardiovascular disease diagnosis, binary indicator for any prior diabetes diagnosis, binary indicator for any prior COPD or asthma diagnosis, and/or binary indicator for any prior kidney disease diagnosis. As shown in the tables 302A, and 306A, and 308 some of the blood test results and/or features can be associated with an identifier, such as, a code from Logical Observation Identifiers Names and Codes (LOINC®).

FIG. 3B depicts non-limiting test results and/or features in a metabolic panel table 302B, a lipid panel table 304, and a complete blood count panel table 306B. These features and any of the features described herein can be derived from other sources, such as a bodily sample (such as a hair, tissue, urine, or saliva sample). As described herein, an advantage to making predictions based on metabolic, lipid, and complete blood count panels is that they can be routine tests for many patients. As shown in the metabolic panel table 302B, metabolic blood test results and/or features can include, but are not limited to, a glucose, calcium, sodium, potassium, bicarbonate, chloride, blood urea nitrogen, creatinine, albumin, total protein, alanine aminotransferase, aspartate aminotransferase, alkaline phosphatase, and/or bilirubin test result and/or feature. As shown in the lipid panel table 304, lipid blood test results and/or features can include, but are not limited to, a total cholesterol (CHOL), high density cholesterol (HDL), low density cholesterol (LDL), and/or triglyceride (TRIG) test result and/or feature. As shown in the complete blood count panel table 306B, complete blood count test results and/or features can include, but are not limited to, a hemoglobin (HGB), hematocrit (HCT), white blood cell count (WBC), red blood cell count (RBC), and/or platelets (PLT) test result and/or feature. As shown in the tables 302B, 304, 306B, each blood test result and/or feature can be associated with an identifier, such as, a code from LOINC. FIGS. 3B and 3C depict additional non-limiting test results and/or features in the table 308A, 308B, 308C.

In some embodiments, the machine learning prediction system 104 can perform binary classification tasks to predict cohort versus control groups using the features described herein (such as the features of FIGS. 3A, 3B, 3C, and/or 3D) using machine learning. The machine learning prediction system 104 can use a Gradient Boosting Decision Tree (GBDT) algorithm/framework (such as Light Gradient-Boosting Machine) to train a model. Evaluation can be performed on a stratified held-out test set that consists of a portion (such as 40%) of the full dataset for each cohort and control pair.

FIG. 4 depicts non-limiting diseases and conditions associated with patients and/or laboratory tests in tables 402, 404, 406. The machine learning prediction system 104 can query the data from the data lake 140 to determine the one or more diseases in the first table 402. As shown in the first table 402, the machine learning prediction system 104 can query the data lake 140 for one or more diagnosis codes (such as an International Statistical Classification of Diseases code) and determine patients associated with specific diagnosis codes. The machine learning prediction system 104 can determine diseases, such as, but not limited to, preterm labor, Parkinson's disease, juvenile rheumatoid arthritis, Alzheimer's disease, sleep apnea, osteoporosis with or without pathological fracture, breast cancer, endometriosis, seropositive rheumatoid arthritis, seronegative rheumatoid arthritis, shingles, pulmonary hypertension, colorectal cancer, Clostridioides difficile (C. Diff), Crohn's disease, systemic lupus erythematosus, celiac disease, colitis, epilepsy, basal cell carcinoma, prostate cancer, Metabolic Associated Fatty Liver disease (MAFLD), Iron Deficiency Anemia, Elevated C-Reactive Protein (CRP), Type 1 diabetes, Type 2 diabetes, prediabetes, autoimmune diseases, cardiovascular diseases, respiratory diseases (including asthma and chronic obstructive pulmonary disease), infectious diseases, metabolic disorders, neurodegenerative conditions, and other disease/conditions, such as any other disease/condition for which blood-based biomarkers provide predictive value.

The machine learning prediction system 104 can query the data from the data lake 140 to determine the one or more diseases and laboratory tests in the second table 404. As shown in the second table 404, the machine learning prediction system 104 can query the data lake 140 for one or more diagnosis codes and determine laboratory tests associated with specific diagnosis codes. The machine learning prediction system 104 can determine laboratory tests associated with diseases, such as, but not limited to, glycogen storage disease, cystic fibrosis, Huntington's disease, muscular dystrophy, amyotrophic lateral scoliosis, polycystic kidney disease, spinal muscular atrophy, Wilson's disease, stiff person syndrome, or Gaucher disease.

The machine learning prediction system 104 can query the data from the data lake 140 to determine the one or more conditions and laboratory tests in the third table 406. As shown in the third table 406, the machine learning prediction system 104 can query the data lake 140 for one or more diagnosis codes and determine laboratory tests associated with specific diagnosis codes. The machine learning prediction system 104 can determine laboratory tests associated with conditions, such as, but not limited to, essential hypertension, diabetes mellitus, hyperlipidemia, atrial fibrillation and flutter, or hypothyroid.

FIG. 5 depicts a workflow 500 illustrating a non-limiting novel clinical insight. As described herein, the machine learning prediction system 104 can diagnose previously undiagnosed diabetes in patients with Parkinson's disease and provide a warning that such patients may be at increased risk for mortality. As described herein, some aspects of the workflow 500 can be performed by the machine learning prediction system 104 and/or by a data analyst.

At one (1), a Parkinson's data set 502 can be determined by querying the data lake 140 with a diagnosis code for Parkinson's disease. The data lake 140 can be queried by the machine learning prediction system 104 and/or by a data analyst. At two (2), from the Parkinson's data set 502, N patients 504 can be determined that have not previously been diagnosed with T2D. In some embodiments, the patients from Parkinson's data set 502 can be queried to determine patients that are not associated with a diagnosis code for T2D. For example, the N patients 504 can be approximately three thousand five hundred unique patients. At three (3), from the N patients 504 that have not previously been diagnosed with T2D, N₁ patients 506 can be determined that are associated with an alive status and N₂ patients 508 can be determined that are associated with a deceased status. For example, the N₁ patients 506 can be approximately twenty-five hundred patients; the N₂ patients 508 can be approximately one thousand patients.

At four (4), the machine learning prediction system 104 can apply a machine learning model, as described herein, to the N₁ patients 506 and the N₂ patients 508 to diagnose X patients 510 and Y patients 512, respectively. Depending on the number of medical encounters used, the machine learning prediction system 104 can predict different numbers of diagnosed patients. For example, the X patients 510 can be approximately one hundred and fifty patients of the N₁ alive patients 506 that are diagnosed with T2D via the machine learning model; the Y patients 512 can be approximately one hundred and fifty patients of the N₂ deceased patients 508 that are diagnosed with T2D via the machine learning model.

Accordingly, the workflow 500 can confirm the novel clinical insight that previously undiagnosed diabetes in patients with Parkinson's disease may be at increased risk for mortality. Specifically, the statistic 514 confirms that the percentage of Y diagnosed patients 512 of the N₂ deceased patients 508 is higher than the percentage of X diagnosed patients 510 of the N₁ alive patients 506. As described herein, a patient that already has a Parkinson's disease diagnosis can receive a further T2D prediction via the machine learning prediction system 104 and the patient and/or the clinician can be provided a warning regarding an increased risk for mortality based at least in part on the additional diabetes prediction.

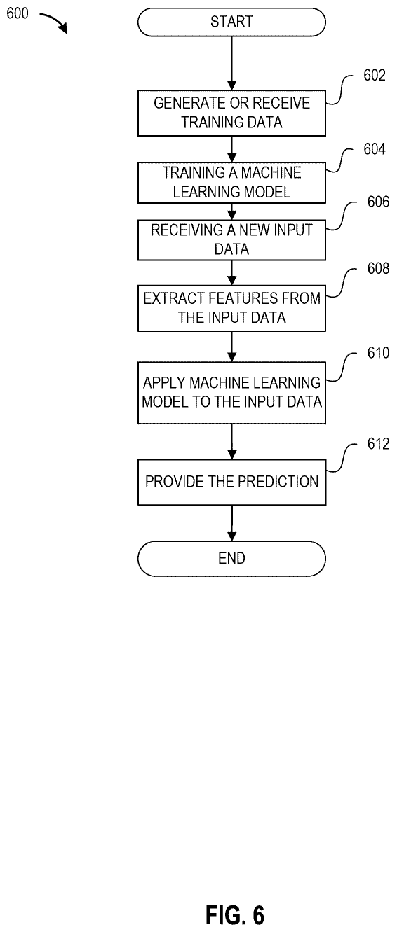

FIG. 6 is a flow chart depicting a method 600 implemented by the machine learning prediction system 104 for making disease or condition predictions. Some aspects of the method 600 may be implemented by components of the machine learning prediction system 104, such as the training server(s) 230 and/or the prediction server(s) 210.

Beginning at block 602, training data can be generated or received. The training server 230 can generate training data based on electronic health records 246 from the data lake 140. As described herein, training data can include test results (such as blood test results) and labels. Each blood test result set can include multiple features. Each blood test result set can include one or more lipid panel test results, one or more metabolic panel test results, and one or more complete blood count test results (or a sub-combination thereof, such as (i) a lipid panel test result and a metabolic panel test result, (ii) a lipid panel test result and a complete blood count test result, (iii) a metabolic panel test result and a complete blood count test result, etc.). Some non-limiting coded features are shown below in Table 1. The training data can include demographics data, such as, but not limited to, age, gender, smoker status. As described herein, each blood test result set can be associated with a particular patient. The training data can include genetic marker data, such as indicators whether a particular genetic marker is present for a patient or not. The training data can include RNA sequence data, such as counts of how much of an RNA sequence is present for particular patients. In some embodiments, the training data can include proteomics data, metabolomics data, and/or epigenomics data. In some embodiments, time can be a feature dimension. Each patient can be associated with either the first label indicating an identification of a disease or condition or the second label indicating the absence of the disease or condition. Some diseases and/or conditions, which can be indicated by labels, can include, but are not limited to, preterm labor, Parkinson's disease, juvenile rheumatoid arthritis, Alzheimer's disease, sleep apnea, osteoporosis with or without pathological fracture, breast cancer, endometriosis, seropositive rheumatoid arthritis, seronegative rheumatoid arthritis, shingles, pulmonary hypertension, colorectal cancer, Clostridioides difficile (C. Diff), Crohn's disease, systemic lupus erythematosus, celiac disease, colitis, epilepsy, basal cell carcinoma, prostate cancer, Metabolic Associated Fatty Liver disease (MAFLD), Iron Deficiency Anemia, Elevated C-Reactive Protein (CRP), Type 1 diabetes, Type 2 diabetes, prediabetes, autoimmune diseases, cardiovascular diseases, respiratory diseases (including asthma and chronic obstructive pulmonary disease), infectious diseases, metabolic disorders, neurodegenerative conditions, and other diseases/conditions, such as any other disease/condition for which blood-based biomarkers provide predictive value.

TABLE 1

| Code | Description |
|---|---|
| CHOL | total cholesterol |
| HDL | high density cholesterol |
| LDL | low density cholesterol |
| TRIG | triglyceride |
| HGB | hemoglobin |
| HCT | hematocrit |
| WBC | white blood cell count |
| RBC | red blood cell count |
| PLT | platelet count |
| GLU | glucose |
| CR | creatinine |
| CA | calcium |
| ALB | albumin |
| NA | sodium |
| PR | total protein |
| K | potassium |
| ALT | alanine aminotransferase |
| CO2 | bicarbonate |
| AST | aspartate aminotransferase |
| CL | chloride |
| ALP SERPL | alkaline phosphatase |
| BUN | blood urea nitrogen |
| BILTDA | bilirubin |
| YEAR | age |
| M | gender |
| N | smoker? |

In some embodiments, some features may not be used for training/prediction. Fast plasma glucose can be present or absent as a feature depending on the disease prediction target. For example, a fast plasma glucose may not be a routine laboratory test, may be relatively expensive, and/or may not be a convenient test for a patient to take. Additional features can be absent, such as, but not limited to, diabetes co-morbidity (such as, hypertension, heart failure, stroke, etc.), some demographics (such as BMI), some blood test results (such as uric acid), urinalysis, and/or test time.

The system allows for the inclusion of additional biomarkers, including but not limited to fasting plasma glucose, uric acid, advanced lipid markers, inflammatory biomarkers (such as C-reactive protein or interleukins), and/or metabolomic or proteomic indicators.

In some embodiments, prior to training, the machine learning prediction system 104 may preprocess, cluster, and/or reduce dimensionality of the training data, thereby optimizing feature selection and/or making training more computationally efficient. Prior to training, an unsupervised or semi-supervised machine learning model may be applied for data clustering, feature extraction, or latent representation learning. The final prediction model may incorporate outputs from one or more of these preprocessing steps. The machine learning prediction system 104 can apply unsupervised learning techniques. The preprocessing techniques can include, but are not limited to, clustering methods (such as k-means, hierarchical clustering, and/or clustering via Gaussian mixture models), dimensionality reduction techniques (such as PCA, t-SNE, and/or UMAP), feature embedding methods (such as utilizing autoencoders and/or variational autoencoders), anomaly detection methods for pre-filtering noisy and/or outlier samples, and/or hybrid modeling approaches that first apply unsupervised learning before supervised model training. The machine learning prediction system 104 can cluster patient-related data that results in multiple data clusters and create the training data based at least in part on a data cluster from the multiple data clusters. Each data cluster of the plurality of data clusters corresponds to a particular disease or condition. For example, the patient-related data can be clustered into data sets for (i) early or late disease onset and/or (ii) each of BRCA1 and BRCA2 genes, which can be utilized for breast cancer prediction.

Additional features that can be included in training data include procedure features (such as data indicating performed medical services and procedures furnished by physicians and other health care professionals, such as CPT-4 codes), medications features, prescriptions features, and/or allergies features.

At block 604, one or more machine learning models can be trained with the training data. The training server 230 can train a machine learning model with the training data. The machine learning model can be configured to predict a likelihood that a patient has or will have a particular disease or condition based at least in part on test data for the patient. The training server 230 can use supervised machine learning algorithms to perform the training. The training server 230 can use machine learning algorithms and/or models such as, but not limited to, Naïve Bayes classifier, a logistic regression model, an artificial neural network, a gradient-boosted trees model (such as, but not limited to, an XGBoost Trees model), a decision tree model, a random forest model, a generalized linear model, a deep learning model (such as, but not limited to, a Keras model), and/or an ensemble model/algorithm (such as, but not limited to, H20 AutoML), a transformer model (such as a model that can process tabular data), a reinforcement learning model, a self-supervised learning models, hybrid architectures combining rule-based systems and machine learning, and any other machine learning model that can process laboratory test results for diagnostic prediction. As described herein, the type of machine learning model or algorithm used can improve accuracy (such as AUC, sensitivity, and/or specificity) over other prediction systems using different models. In some embodiments, the trained machine learning model can include or correspond to a gradient-boosted tree model (such as an XGBoost Trees model), which can achieve better accuracy over some other models. Additionally or alternatively, the trained machine learning model can include or correspond to an ensemble model, which can achieve better accuracy over some other models. In some embodiments, the training servers 230 can train multiple models of different types and select the model based on predictive performance (such as selecting the model with the best predictive performance or selecting the model that satisfies a predictive performance threshold). Moreover, the training server 230 can train multiple models where respective models can be trained to predict particular diseases or conditions.

A model for each disease can be evaluated on a held-out test set (such as 40%) stratified on the diagnosis target variable. Area under receiver operator curve (ROC-AUC), sensitivity, specificity, and positive predictive value (PPV) can be calculated from the test set using the trained models. The following table, Table 2, shows the performance metrics of a held-out test set.

TABLE 2

| | | Cohort | | | | | | |
|---|---|---|---|---|---|---|---|---|
| | Min | Train | Control | Cohort | Control | ROC | Sens @ 90% | PPV @ 90% |
| | Age | Size | Train | Test | Test | AUC | Specificity | Specificity |
| Chronic Kidney | 40 | 12788 | 50596 | 8525 | 33732 | 0.93 | 0.82 | 0.67 |
| Disease | | | | | | | | |
| Type 2 Diabetes | 20 | 10270 | 91713 | 6846 | 61144 | 0.89 | 0.69 | 0.44 |
| Heart Failure | 50 | 12475 | 150420 | 8317 | 100281 | 0.91 | 0.71 | 0.37 |
| TIA/Ischemic | 50 | 4615 | 11372 | 3067 | 74250 | 0.84 | 0.52 | 0.18 |
| Stroke | | | | | | | | |
| Lung Cancer (ever | 50 | 1354 | 47235 | 902 | 31491 | 0.86 | 0.59 | 0.15 |
| smoker) | | | | | | | | |
| Osteoarthritis | 50 | 14623 | 114640 | 9748 | 76428 | 0.81 | 0.45 | 0.38 |
| Colorectal | 45 | 1009 | 370140 | 433 | 158632 | 0.83 | 0.58 | 0.013 |
| Cancer | | | | | | | | |
| C. Diff | 18 | 1780 | 452707 | 1186 | 301806 | 0.86 | 0.61 | 0.023 |
| MAFLD | 18 | 6839 | 497863 | 4559 | 331909 | 0.87 | 0.63 | 0.08 |
| Iron Deficiency | 18 | 8020 | 583234 | 5347 | 388823 | 0.91 | 0.78 | 0.1 |
| anemia | | | | | | | | |
| Elevated CRP | 49 | 9581 | 29242 | 6388 | 19494 | 0.84 | 0.49 | 0.61 |
| Rheumatoid | 20 | 2199 | 48031 | 1466 | 32021 | 0.77 | 0.4 | 0.16 |
| Arthritis | | | | | | | | |
| Prostate Cancer | 50 | 1319 | 26490 | 1978 | 39734 | 0.83 | 0.49 | 0.20 |
| Hypothyroidism | 18 | 14110 | 59688 | 9407 | 146569 | 0.79 | 0.46 | 0.14 |
| Kidney Ureter | 20 | 5000 | 108970 | 7500 | 163454 | 0.76 | 0.4 | 0.15 |
| Stone | | | | | | | | |
| Epilepsy | 18 | 1954 | 115894 | 1302 | 77264 | 0.76 | 0.42 | 0.05 |
| Breast Cancer | 40 | 1385 | 16031 | 924 | 10687 | 0.76 | 0.36 | 0.24 |

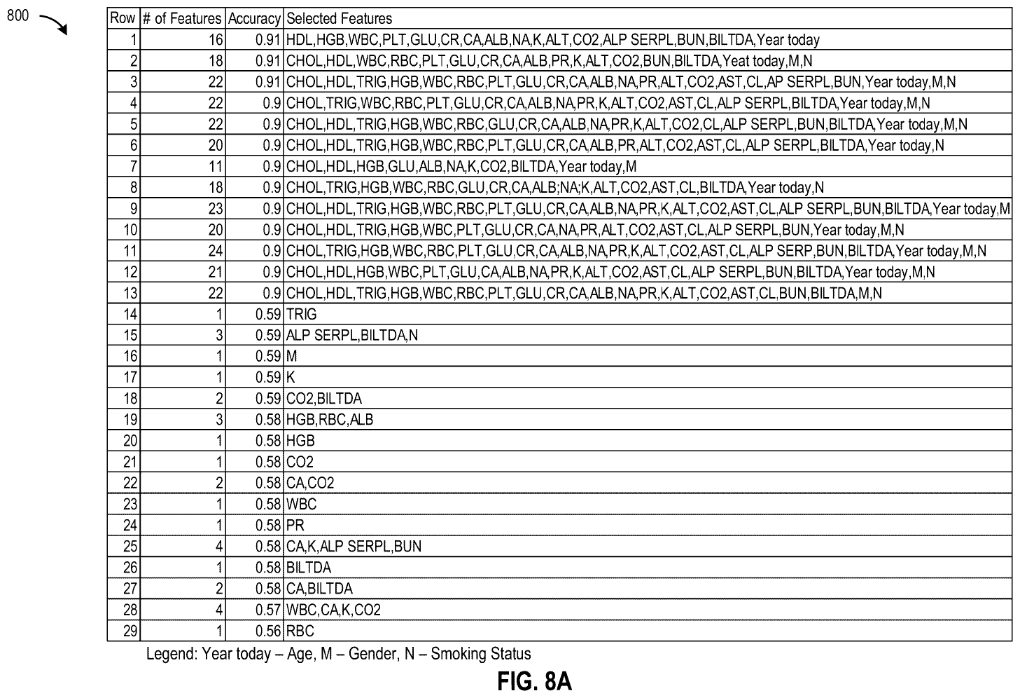

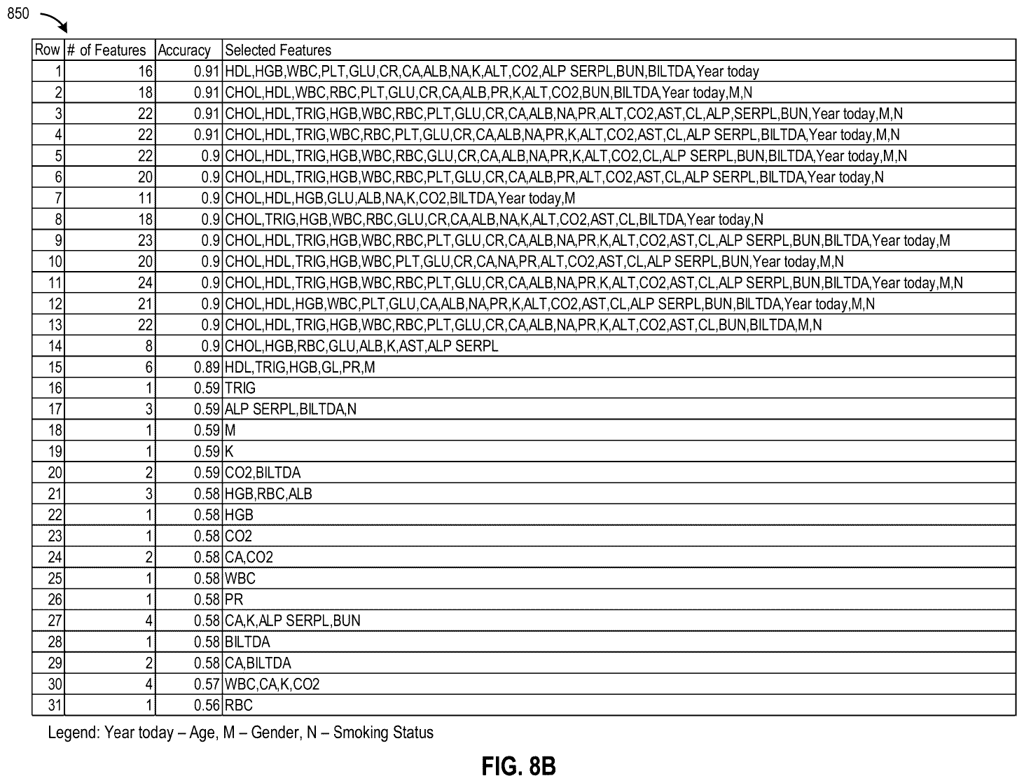

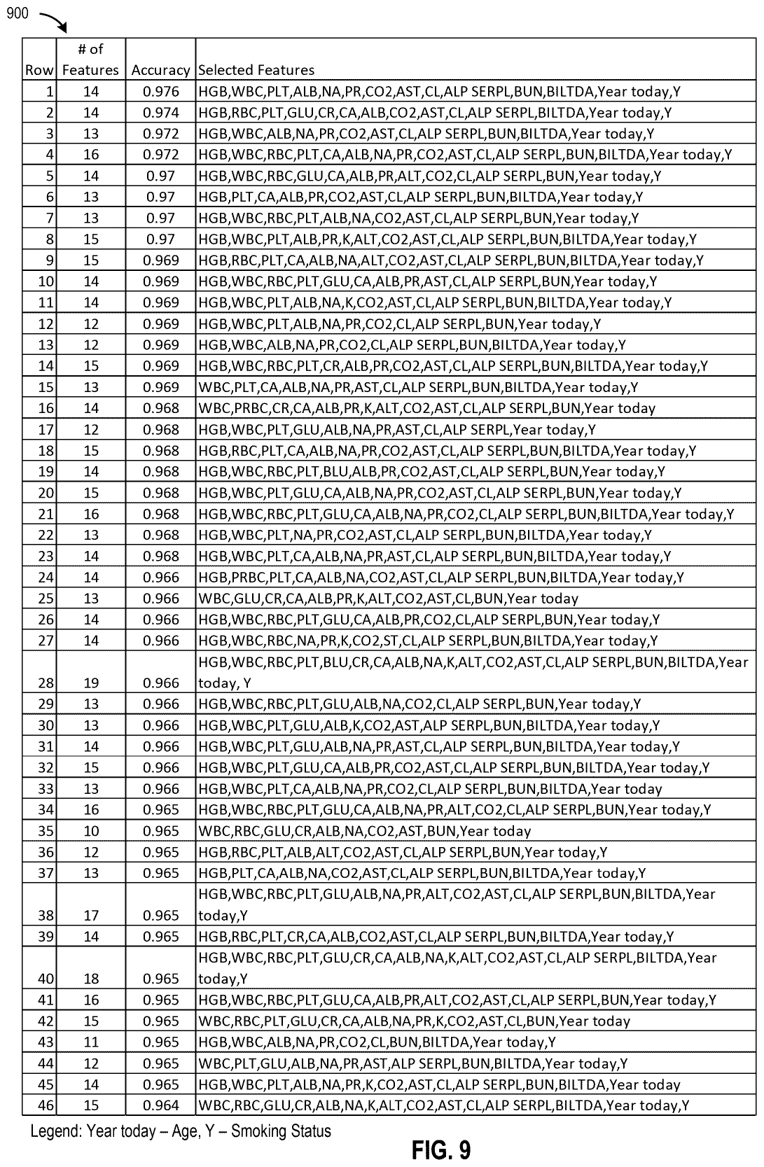

As described herein, the particular features used for prediction training can improve accuracy (such as AUC, sensitivity, and/or specificity) over other prediction systems using different features. The training server 230 and/or a data analyst can identify a subset of features that has sufficient and/or improved accuracy. For example, a subset of features (such as the features in Table 1) can be identified. Additional details regarding particular features and/or feature selection for machine learning based diagnostic prediction training are described herein, such as with respect to FIGS. 8A-8B and 9. In some embodiments, some models can be trained with a subset of features and still have sufficient accuracy. The training server 230 can identify different subsets of features as predictive. For example, a risk of preterm labor can be predicted based on a first feature corresponding to a hemoglobin value, a second feature corresponding to a white blood cell count value, a third feature corresponding to a platelet count value, a fourth feature corresponding to an albumin value, a fifth feature corresponding to a sodium value, a sixth feature corresponding to a protein value, a seventh feature corresponding to a bicarbonate value, an eighth feature corresponding to an aspartate aminotransferase value, a ninth feature corresponding to a chloride value, a tenth feature corresponding to an alkaline phosphatase value, an eleventh feature corresponding to a blood urea nitrogen value, a twelfth feature corresponding to a bilirubin value, a thirteenth feature corresponding to an age value (shown as “Year today” in the table 900 of FIG. 9, which can be a current age value), a fourteenth feature corresponding to a smoker value (shown as “Y” in the table 900 of FIG. 9). In other embodiments, different features can be used for training/prediction of preterm labor, such as complete blood count features (such as the features from the complete blood count panel table(s) 306A and 306B), metabolic features (such as the features from the metabolic panel table(s) 302A and 302B), an age value, and a smoker value.

Aspects of the present disclosure (such as usage of the particular features in machine learning described herein) can allow some embodiments to have improved accuracy (such as sensitivity and/or accuracy above 80 or 90 percent) for predicting diseases or conditions, such as, but not limited to, preterm labor, Parkinson's disease, juvenile rheumatoid arthritis, Alzheimer's disease, sleep apnea, osteoporosis with or without pathological fracture, breast cancer, endometriosis, seropositive rheumatoid arthritis, seronegative rheumatoid arthritis, shingles, pulmonary hypertension, colorectal cancer, Clostridioides difficile (C. Diff), Crohn's disease, systemic lupus erythematosus, celiac disease, colitis, epilepsy, basal cell carcinoma, prostate cancer, Metabolic Associated Fatty Liver disease (MAFLD), Iron Deficiency Anemia, Elevated C-Reactive Protein (CRP), Type 1 diabetes, Type 2 diabetes, prediabetes, autoimmune diseases, cardiovascular diseases, respiratory diseases (including asthma and chronic obstructive pulmonary disease), infectious diseases, metabolic disorders, neurodegenerative conditions, and other diseases/conditions, such as diseases/conditions for which blood-based biomarkers provide predictive value. In some embodiments, preterm labor can be predicted earlier using features described herein (such as including, but not limited to, the blood test results) for a preterm labor diagnosed cohort up to a threshold period (such as 90 days) before first diagnosis. For example, preterm labor can be predicted where the laboratory tests were performed 1-30 days, 31-60 days, and/or 61-90 days before a diagnosis date in the training data.

In some embodiments, the machine learning models can be trained to predict a single class (whether the disease or condition is or will be present). Accordingly, the systems and methods described herein can be different from other systems that perform multiclass prediction (predicting multiple classes, such as healthy, prediabetic, and diabetic). In some embodiments, a single class prediction approach can lead to higher accuracy than other multiclass prediction models.

In some embodiments, the machine learning models can go through retrospective validation where there is a training period and a trial period. The retrospective validation can be applied to machine learning models for diseases, such as, but not limited to, heart failure, stroke/TIA, lung cancer, and/or colon cancer. The training period is defined to be the period prior to a particular cutoff date, and the “trial period” can be defined from that cutoff date and onwards. In the “trial period,” for each patient, the CBC and CMP lab results that are closest to the beginning of the “trial period” can be taken and followed those results retrospectively for a period (such as a maximum of two years). Degrees of risk (such as moderate risk, high risk, and very high risk) cutoffs can be defined to correspond to particular thresholds (such as 0.7, 0.9, and 0.98) of specificity based on the cutoffs from the model trained on the training data.

As described herein, the training server 230 can train a machine learning model based on training data regarding performed medical services and procedures furnished by physicians and other health care professionals (such as CPT-4 codes), medications, prescriptions, and/or allergies.

At block 606, new input data can be received. The prediction server 210 can receive new input data for a patient. New input data, can include, but is not limited to, test data (such as a blood test result), demographics data, proteomics data, metabolomics data, epigenomics data, genetic data, and/or RNA data.

At block 608, features can be extracted from the input data. The prediction server 210 can extract features from the input data. The prediction server 210 can generate vector data from the input data. As described herein, some extracted features can include, but are not limited to, the features in Table 1 above or a subset of features. The prediction server 210 can generate an additional feature for the patient corresponding to a genetic marker value (which can be a 0 or 1, for example). The prediction server 210 can generate an additional feature for the patient corresponding to an RNA sequence value (such as a count of much of an RNA sequence is present for the patient).

As described herein, the particular features used for diagnostic prediction can improve accuracy (such as AUC, sensitivity, and/or specificity) over other prediction systems using different features. As described herein, a subset of features (such as the features in Table 1) can be identified. In some embodiments, some models can still accurately make predictions at inference time even if some of the input data (such as values for some features) may be missing. A missing value can be a null value.

In some embodiments, additional features extracted from the input data can include procedure features (such as data indicating performed medical services and procedures furnished by physicians and other health care professionals, such as CPT-4 codes), medications features, prescriptions features, and/or allergies features.

At block 610, the trained machine learning model can be applied to the input data. The prediction server 210 can apply the trained machine learning model to the input data. The prediction server 210 can execute the machine learning model, where the machine learning model receives the features as input and the machine learning model outputs a score that reflects a likelihood of that the patient has or will have the disease or condition (such as, but not limited to, diabetes, prediabetes, or preterm labor). The score can indicate a probability, a risk category, and/or a classification. As described herein, additional features can include genetic data and/or RNA sequence data. In some embodiments, the prediction server 210 can store the prediction in the prediction data storage 214. In some embodiments, the prediction server 210 can convert the score into another format, such as a Boolean value. The prediction server 210 can provide a Boolean value if the score value satisfies a threshold value.

As described herein, the prediction server 210 can allow missing/null values. The prediction server 210 can apply/utilize data imputation techniques to make predictions, such as, but not limited to, statistical imputation, machine learning-based predictive imputations, synthetic data generation, and/or dynamic real-time feature estimation based on ensemble models. These approaches can enable robust prediction accuracy even in the presence of incomplete or missing data.

At block 612, the prediction can be provided. The machine learning prediction system 104 can provide the prediction to the client device 202. In some embodiments, the client device 202 can be associated with a healthcare provider and the client device 202 can receive prediction result(s). The machine learning prediction system 104 can output the score or the prediction to the client device 202. In some embodiments, the machine learning prediction system 104 can output the score or the prediction to memory and/or data storage. Additionally or alternatively, the client device 202 can be a user computing device. In some embodiments, the machine learning prediction system 104 can output the prediction result(s) to a graphical user interface.

In some embodiments, the method 600 can include treatment. For example, after a clinician receives the score from the machine learning prediction system 104, the clinician can treat the disease or condition. For example, a patient can be treated for the disease or condition (such as, but not limited to, diabetes, prediabetes, or preterm labor) based on the prediction. A patient can be prescribed medication. For example, a patient can be prescribed medication to treat diabetes or prediabetes or to lower the risk of preterm labor.

### Type 2 Diabetes Prediction Embodiments

T2D is a health condition where the body's blood sugar levels become unbalanced because it does not respond well to insulin. This can lead to other health problems such as heart disease, stroke, and kidney damage. In 2022, 38 million Americans have been diagnosed with diabetes. It is estimated that about 8 million Americans might have undiagnosed T2D per year, while only about 1.4 million Americans are diagnosed with T2D per year. The systems and methods described herein can use supervised machine learning to create new tools to check for T2D and predict the presence of T2D early and accurately. The tool can use results from routine blood tests that can be obtained through yearly wellness checkups. Compared to other T2D tests that are currently in use, like hemoglobin A1C and fasting plasma glucose, the improved diabetes prediction tools can have better sensitivity (88.3%, a measure of how good the test is at finding people with T2D) and specificity (90.0%, indicates how accurate the test is when saying a person has T2D).

Because some of the T2D tools described herein can be based on blood tests routinely performed during the initial diagnosis process of most diseases, the extra insights these tools can provide can prevent the prescription of drugs that increase sugar levels, as this can be fatal to undiagnosed T2D individuals. Certain steroids, antipsychotics, antiretrovirals, lipid-lowering agents, and others are examples of drugs that are typically not prescribed to diagnosed T2D individuals. Chemotherapy treatment is also reported to increase blood sugar levels in diabetics.

The machine learning prediction system 104 can use the data lake 140. The data lake 140 can include millions of de-identified, healthcare-related datapoints (lab test results, images, doctor's notes, etc.), which can be used for analysis using supervised machine learning. The systems and methods described herein, such as the machine learning prediction system 104, can utilize lab results from routine blood tests (such as the metabolic panel table(s) 302A and 302B, the lipid panel table 304, and/or the complete blood count panel table(s) 306A and 306B of FIGS. 3A and 3B) as features in a supervised machine learning model that diagnoses T2D with high sensitivity and specificity without the need for an A1C test (such as one provided via a separate doctor's visit for a hemoglobin A1C test). The T2D prediction ML models described herein can use the lipid, CBC, and metabolic panels (or some combination thereof). Moreover, the T2D prediction ML models described herein can incorporate all three panels without requiring missing feature value imputation. As a result, the supervised machine learning algorithms described herein can achieve improved (such as a first-in-class) T2D diagnostic tools with sensitivity and specificity of approximately 90%.

An individual undergoing a routine blood test, which can be part of the yearly wellness exam, can make use of the T2D diagnostic tools described herein. Based on the blood test results, specifically the lipid, CBC, and/or metabolic panels, the systems and methods described herein can give an important added insight of high sensitivity (low false positive rate) T2D diagnosis without the need for traditional, but low sensitivity, T2D diagnostic tests like A1C or fasting plasma glucose (FPG). Current T2D diagnostic tests, such as A1C, FPG, or oral glucose tolerance test (OGTT), require additional doctor's visits and patient expense. The T2D diagnostic tools described herein based on routine lab tests (independent of tests such as the A1C test) can have broader use and utility, which can also address under diagnosed diabetes cases. This can also address the unmet need of a more rapid and accurate way to detect diabetes earlier. Supervised machine learning can be performed using routine annual blood-based lab tests, such as test results from the lipid, CBC, and metabolic panels (described herein, such as, with respect to FIGS. 3A and 3B), as features to diagnose T2D.

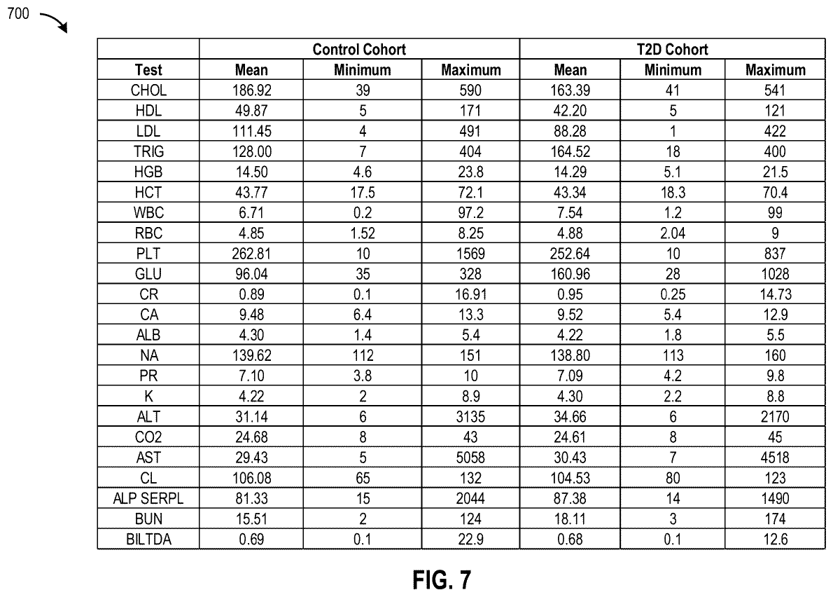

FIG. 7 depicts the test results of diabetes and control cohorts. As shown, except for glucose (GLU), the values (such as mean, minimum, and maximum) of each test result are not dissimilar. Accordingly, the correlation of these tests, taken as one or more combinations, to T2D diagnosis is neither apparent nor obvious. De-identified blood test results from T2D (ICD10 code E11.*) patients and control cohort (not E11.*) can be received and/or identified from the data lake 140. The downloaded data can be pre-processed with the goal of removing any incomplete blood panel test result for a specific medical encounter from further analysis. A resulting data set can include non-imputed lab test results from unique medical encounters (such as over ˜20,000) for either T2D or control cohorts (such as total samples with complete lab test results ˜50,000).

For cohort stratification, a first threshold (such as A1C levels>6.5) can be used to label T2D diagnosis while a second threshold (such as A1C levels≤6.5) can indicate normal control. The thresholds can be derived from current guidelines. Based on a review of different A1C levels to optimized T2D diagnosis, it can be determined that T2D A1C≥6.1 can provide improved sensitivity and/or specificity values. Following data cleanup and concatenation, control samples (such as approximately 24,000-26,000 samples) and samples associated with T2D (such as approximately 19,000-24,000 samples) can be identified. Linear correlation analysis can be performed to observe correlations, such as correlations between CHOL to LDL and HGB to HCT. Consequently, some features, such as, but not limited to, features LDL and HCT, can be excluded from further supervised machine learning analyses. Next, preliminary feature selection analysis (such as random forest-based preliminary feature selection analysis) can be performed to determine accuracy for T2D diagnosis.

FIGS. 8A-8B depict the results of automated feature selection for diabetes diagnosis ranked by accuracy. As shown in the tables 800, 850 of FIG. 8A-8B, each row can indicate number of features used, accuracy, and selected features. The results of the analysis can reveal that it can be advantageous to use some of the blood test results together with age, gender, and smoker status (as shown by rows 1-13 of the table 800 of FIG. 8A or rows 1-15 of the table 850 of FIG. 8B). T2D diagnosis accuracy can change when using fewer features (as shown by rows 14-29 of the table 800 of FIG. 8A or the rows 16-31 of the table 850 of FIG. 8B).

In some embodiments, numeric outlier analysis can be performed for the lab test results, such as flagging values outside the interquartile range (IQR). In some embodiments, approximately 4% were outside the IQR range and optimal sensitivity and specificity were obtained when these outlier values were replaced by moving average value replacement (lookbehind/ahead=1000).

A machine learning model can be refined by using a set of features (such as the 24 features of row 11 of the table 800 of FIG. 8A or row 11 of the table 850 of FIG. 8B) and an age range (such as 8 to 75 years old) from a training dataset (such as a 90% partition). The machine learning model can be refined using the features and k=n-fold (such as 10-fold) cross validation on the training dataset (such as a 90% partition). In some embodiments, an XGBoost Trees model can diagnose T2D, yielding metrics, such as, but not limited to: AUC=˜96%, sensitivity=˜ 91%, and specificity=˜ 88-89%. The same model can be validated on an independent test dataset (such as a 10% partition) resulting in a sensitivity of ˜88-91% and specificity of ˜90%. The sensitivity and specificity of novel T2D diagnostic tool(s) described herein can surpass those of the A1C test (74.0% and 87.2%, respectively) and the fasting plasma glucose (FPG) test (82.3% and 89.4%, respectively).

In some embodiments, the novel diagnostic tool(s) described herein can be positioned to improve undiagnosed T2D detection because (i) some aspects of the T2D insights described herein can be derived from blood test panels taken during yearly wellness checkups and, thus, may not require extra time and effort from patients, and/or (ii) the novel algorithms described herein can result in improved (such as a best-in-class) population-level screening tool with high sensitivity (fewer false negative) and specificity (fewer false positives) compared to A1C and FPG tests. The diagnostic solutions described herein are also amenable to incomplete lab test results, and the machine learning algorithms described herein can be used to train models that use less features and still have high accuracy as needed.

For example, using the features that gave the highest accuracy (such as row 1 of the table 850 of FIG. 8B) from random forest-based preliminary feature selection to diagnose T2D from the validation data set resulted in sensitivity and specificity of ˜91% and ˜89%, respectively. Using the 8 and 6 features that gave an accuracy of 90% and 89%, respectively, (such as rows 14 and 15 of the table 850 of FIG. 8B, respectively) resulted in sensitivity and specificity of (˜89.3%, ˜87%) and (˜88% and ˜85%), respectively. Using a 3-feature T2D diagnosis model (such as a model with GLU, CA, and K features-all members of the metabolic panel) still yielded a sensitivity and specificity of ˜89% and ˜84%, which can further validate the tractability the T2D diagnostic tool(s) with fewer lab test features, as described herein.

In some embodiments, streamlined T2D diagnosis can be based on routine blood tests. Relatively higher sensitivity and higher specificity can mean relatively lower false positive and lower false negative results. Deployment of the tool(s) described herein can significantly reduce misdiagnosis of T2D and the number of undiagnosed cases. Earlier diagnosis can lead to better patient outcomes. The diagnostics described herein can be included as part of yearly wellness checkups without the additional overhead associated with new lab tests. Identification of T2D patients in early stages of the disease can open unique research opportunities to test new therapeutics and preventative measures. There has been a surge of diabetes cases due to the increased proliferation of poor dietary habits and inactive lifestyles. Corporations and healthcare insurance providers who invest in yearly wellness checkups can provide important health insights with the tool(s) described herein.

The tool(s) described herein can be used to develop pharmaceuticals, such as during clinical trials. Streamlined identification of undiagnosed T2D cohorts could lead to more efficient enrollment and faster clinical trial start times. Addition of the diagnostic tool(s) described herein to clinical trials can help monitor the emergence of T2D as a potential negative side-effect (or the lowering of T2D probability as a potential positive side-effect) based on routine blood tests used to monitor patient health during the study. The effect of undiagnosed/untreated T2D versus treated T2D on a therapeutic profile of a candidate drug can be monitored.

In some embodiments, diabetes-risk gene mutations can be added as a feature to the model. In some embodiments, BMI can be added as a feature to the model. In some embodiments, the tools described herein can be stratified by age, gender, ethnicity. For example, training a supervised machine learning model with inclusion criteria of 18-45 years old can result in increased specificity (˜95%) and decreased sensitivity (˜80%) when a validation dataset is used. As described herein, it was not obvious, nor easily discernable how the blood test features (such as the 23 features described herein) are correlated to T2D diagnosis. The systems and methods described herein can result in significant increase in sensitivity and specificity. Moreover, the systems and methods described herein can result in improved patient convenience as well as improved standard of care

The systems and methods described herein can apply supervised machine learning to T2D diagnosis in novel ways. The features used to train machine learning models can be complete and thus machine learning missing value imputation may not be needed. Because of this, the diagnostic tools described herein can have relatively high sensitivity (˜90%) and specificity (˜88%). Moreover, if a patient's blood test results are incomplete, the diagnostic tool(s) described herein can use different combinations of features while maintaining relatively high accuracy.

### Preterm Labor Prediction Embodiments

FIG. 9 depicts the results of automated feature selection for preterm labor prediction ranked by accuracy. As shown in the table 900 of FIG. 9, each row can indicate number of features used, accuracy, and selected features. The results of the analysis can reveal that it can be advantageous to use some of the blood test results together with age and smoker status.

### Implementation Details

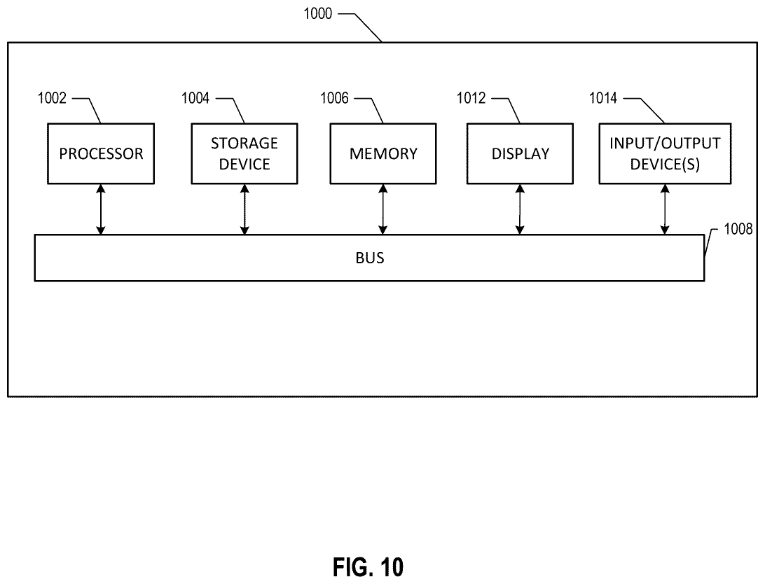

FIG. 10 is a block diagram that illustrates example components of a computing device 1000. The computing device 1000 can implement aspects of the present disclosure. Using FIG. 2 as an example, the prediction server 210 or the training server 230 of FIG. 2 can be implemented in a similar manner as the computing device 1000. The computing device 1000 can communicate with other computing devices.

The computing device 1000 can include a hardware processor 1002, a data storage device 1004, a memory device 1006, a bus 1008, a display 1012, and one or more input/output devices 1014. The hardware processor 1002 can also be implemented as a combination of computing devices, e.g., a combination of a digital signal processor and a microprocessor, a plurality of microprocessors, one or more microprocessors in conjunction with a digital signal processor, or any other such configuration. The hardware processor 1002 can be configured, among other things, to execute instructions to perform one or more functions. The data storage device 1004 can include a magnetic disk, optical disk, solid state drive, or flash drive, etc., and is provided and coupled to the bus 1008 for storing information and instructions. The memory device 1006 can include one or more memory devices that store data, such as, without limitation, random access memory (RAM) and read-only memory (ROM). The computing device 1000 may be coupled via the bus 1008 to the display 1012, such as an LCD display or touch screen, for displaying information to a user, such as an engineer. The computing device 1000 may be coupled via the bus 1008 to one or more input/output devices 1014. The input device 1014 can include, but is not limited to, a keyboard, mouse, digital pen, microphone, or touch screen.

A machine learning application may be stored on the memory device 1006 and executed as a service by the hardware processor 1002. In some embodiments, the machine learning application may implement various aspects of the present disclosure. For example, the machine learning application may train a machine learning model configured to predict a diagnosis.

It is to be understood that not necessarily all objects or advantages may be achieved in accordance with any particular embodiment described herein. Certain embodiments may be configured to operate in a manner that achieves or optimizes one advantage or group of advantages as taught herein without necessarily achieving other objects or advantages as may be taught or suggested herein.

Many other variations than those described herein will be apparent from this disclosure. For example, depending on the embodiment, certain acts, events, or functions of any of the algorithms described herein can be performed in a different sequence, can be added, merged, or left out altogether (e.g., not all described acts or events are necessary for the practice of the algorithms). Moreover, in certain embodiments, acts or events can be performed concurrently, e.g., through multi-threaded processing, interrupt processing, or multiple processors or processor cores or on other parallel architectures, rather than sequentially. In addition, different tasks or processes can be performed by different machines and/or computing systems that can function together.

The various illustrative logical blocks and modules described in connection with the embodiments disclosed herein can be implemented or performed by a machine, such as a processing unit or processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable device that performs logic operations without processing computer-executable instructions. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor may also include primarily analog components. For example, some or all of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

Conditional language used herein, such as, among others, “can,” “might,” “may,” “e.g.,” “for example,” and the like, unless specifically stated otherwise, or otherwise understood within the context as used, is generally intended to convey that certain embodiments include, while other embodiments do not include, certain features, elements, or states. Thus, such conditional language is not generally intended to imply that features, elements or states are in any way required for one or more embodiments.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Such disjunctive language is not generally intended to, and should not, imply that certain embodiments require at least one of X, at least one of Y, and at least one of Z to each be present. Thus, the term “or” is used in its inclusive sense (and not in its exclusive sense) so that when used, for example, to connect a list of elements, the term “or” means one, some, or all of the elements in the list.

Any process descriptions, elements or blocks in the flow diagrams described herein and/or depicted in the attached figures should be understood as potentially representing modules, segments, or portions of code which include one or more executable instructions for implementing specific logical functions or elements in the process. Alternate implementations are included within the scope of the embodiments described herein in which elements or functions may be deleted, executed out of order from that shown, or discussed, including substantially concurrently or in reverse order, depending on the functionality involved.

The term “a” as used herein should be given an inclusive rather than exclusive interpretation. For example, unless specifically noted, the term “a” should not be understood to mean “exactly one” or “one and only one”; instead, the term “a” means “one or more” or “at least one,” whether used in the claims or elsewhere in the specification and regardless of uses of quantifiers such as “at least one,” “one or more,” or “a plurality” elsewhere in the claims or specification.

The terms “comprising,” “including,” “having,” and the like are synonymous and are used inclusively, in an open-ended fashion, and do not exclude additional elements, features, acts, operations, and so forth.

While the above detailed description has shown, described, and pointed out novel features as applied to various embodiments, it will be understood that various omissions, substitutions, and changes in the form and details of the devices or algorithms illustrated can be made without departing from the spirit of the disclosure. As will be recognized, certain embodiments described herein can be embodied within a form that does not provide all of the features and benefits set forth herein, as some features can be used or practiced separately from others.

What is claimed is:

1. A method comprising:
receive training data comprising (i) a plurality of blood test results, (ii) a first label, and (iii) a second label,
wherein each blood test result set, from the plurality of blood test results, comprises a plurality of features,
wherein each blood test result set, from the plurality of blood test results, comprises a lipid panel test result, a metabolic panel test result, and a complete blood count test result,
wherein each blood test result set, from the plurality of blood test results, is associated with a particular patient, and
wherein each patient is associated with either (i) the first label indicating an identification of a disease or a condition or (ii) the second label indicating absence of the disease or the condition;
training a machine learning model with the training data, wherein the machine learning model is configured to predict a likelihood that a patient has or will have the disease or the condition based at least in part on a new blood test result set for the patient;
receiving a first blood test result set for a first patient;
extracting a first plurality of features from the first blood test result set;
applying the machine learning model to the first plurality of features as first input, wherein the machine learning model outputs a score that reflects a first likelihood that the first patient has or will have the disease or the condition; and
outputting the score.

2. The method of claim 1, wherein the disease or condition comprises at least one of: preterm labor, Parkinson's disease, juvenile rheumatoid arthritis, Alzheimer's disease, sleep apnea, osteoporosis with or without pathological fracture, breast cancer, endometriosis, seropositive rheumatoid arthritis, seronegative rheumatoid arthritis, shingles, pulmonary hypertension, colon cancer, Crohn's disease, systemic lupus erythematosus, celiac disease, colitis, epilepsy, basal cell carcinoma, prostate cancer, Type 1 diabetes, Type 2 diabetes, prediabetes, autoimmune disease, cardiovascular disease, respiratory disease, asthma, chronic obstructive pulmonary disease, infectious disease, metabolic disorder, or neurodegenerative condition.

3. The method of claim 1, wherein a fast plasma glucose feature is absent from the first plurality of features.

4. The method of claim 1, further comprising:
clustering patient-related data that results in a plurality of data clusters; and
creating the training data based at least in part on a data cluster from the plurality of data clusters.

5. The method of claim 1, wherein the training data further comprises genetic marker data, further comprising:
generating an additional feature for the patient corresponding to a genetic marker value, wherein the machine learning model receives the additional feature as second input.

6. The method of claim 1, wherein the training data further comprises ribonucleic acid sequence data, further comprising:
generating an additional feature for the patient corresponding to a ribonucleic acid sequence value, wherein the machine learning model receives the additional feature as second input.

7. A method comprising:
receiving training data comprising (i) a plurality of blood test results, (ii) a first label, and (iii) a second label,
wherein each blood test result set, from the plurality of blood test results, comprises a plurality of features,
wherein each blood test result set, from the plurality of blood test results, comprises a metabolic panel test result and a complete blood count test result,
wherein each blood test result set, from the plurality of blood test results, is associated with a particular patient, and
wherein each patient is associated with either (i) the first label indicating an identification of a disease or a condition or (ii) the second label indicating absence of the disease or the condition;
training a machine learning model with the training data, wherein the machine learning model is configured to predict a likelihood that a patient has or will have the disease or the condition based at least in part on a new blood test result set for the patient;
receiving a first blood test result set for a first patient;
extracting a first plurality of features from the first blood test result set;
applying the machine learning model to the first plurality of features as first input, wherein the machine learning model outputs a score that reflects a first likelihood that the first patient has or will have the disease or the condition; and
outputting the score.

8. The method of claim 7, wherein the disease or the condition comprises preterm labor.

9. The method of claim 7, wherein the training data further comprises genetic marker data, further comprising:
generating an additional feature for the patient corresponding to a genetic marker value, wherein the machine learning model receives the additional feature as second input.

10. The method of claim 7, wherein the training data further comprises ribonucleic acid sequence data, further comprising:
generating an additional feature for the patient corresponding to a ribonucleic acid sequence value, wherein the machine learning model receives the additional feature as second input.

11. The method of claim 7, wherein the machine learning model comprises at least one of: a gradient-boosted tree, an ensemble model, a deep neural network, a transformer model, a reinforcement learning model, or a hybrid architecture combining a rule-based system and a machine learning system.

12. The method of claim 7, further comprising:
clustering patient-related data that results in a plurality of data clusters; and
creating the training data based at least in part on a data cluster from the plurality of data clusters.

13. The method of claim 12, wherein each data cluster of the plurality of data clusters corresponds to a particular disease or condition.

14. A system comprising:
one or more data storage media configured to store specific computer-executable instructions; and
one or more computer hardware processors configured to communicate with the one or more data storage media, wherein the specific computer-executable instructions are configured to cause the one or more computer hardware processors to at least:
receive training data comprising (i) a plurality of blood test results, (ii) a first label, and (iii) a second label,
wherein each blood test result set, from the plurality of blood test results, comprises a plurality of features,
wherein each blood test result set, from the plurality of blood test results, comprises a metabolic panel test result and a complete blood count test result,
wherein each blood test result set, from the plurality of blood test results, is associated with a particular patient, and
wherein each patient is associated with either (i) the first label indicating an identification of a disease or a condition or (ii) the second label indicating absence of the disease or the condition;
train a machine learning model with the training data, wherein the machine learning model is configured to predict a likelihood that a patient has or will have the disease or the condition based at least in part on a new blood test result set for the patient;
receive a first blood test result set for a first patient;
extract a first plurality of features from the first blood test result set;
apply the machine learning model to the first plurality of features as first input, wherein the machine learning model outputs a score that reflects a first likelihood that the first patient has or will have the disease or the condition; and
output the score.

15. The system of claim 14, wherein a fast plasma glucose feature is absent from the first plurality of features.

16. The system of claim 14, wherein the one or more computer hardware processors are configured to execute further computer-executable instructions to at least:
cluster patient-related data that results in a plurality of data clusters; and
create the training data based at least in part on a data cluster from the plurality of data clusters.

17. The system of claim 16, wherein each data cluster of the plurality of data clusters corresponds to a particular disease or condition.

18. The system of claim 14, wherein the training data further comprises genetic marker data, wherein the one or more computer hardware processors are configured to execute further computer-executable instructions to at least:
generate an additional feature for the patient corresponding to a genetic marker value, wherein the machine learning model receives the additional feature as second input.

19. The system of claim 14, wherein the training data further comprises ribonucleic acid sequence data, wherein the one or more computer hardware processors are configured to execute further computer-executable instructions to at least:
generate an additional feature for the patient corresponding to a ribonucleic acid sequence value, wherein the machine learning model receives the additional feature as second input.

20. The system of claim 14, wherein the machine learning model comprises at least one of: a gradient-boosted tree, an ensemble model, a deep neural network, a transformer model, a reinforcement learning model, or a hybrid architecture combining a rule-based system and a machine learning system.
